(19) United States
(12) Patent Application Publication
(10) Pub. No.: US 2009/0196921 A1
(43) Pub. Date: Aug. 6, 2009

(54) Compositions Methods and Kits For Enhancing Immune Response To A Respiratory Condition

(75) Inventors: James Patrick Ebel, Lebanon, OH (US); Lucy Anne Gildea, Cincinnati, OH (US); David Alexander Lawson, Glendale, OH (US); Jeffrey Warren Clymer, Mason, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: The Procter & Gamble Company, Cincinnati, OH (US)

(21) Appl. No.: 12/366,987

(22) Filed: Feb. 6, 2009

Related U.S. Application Data
(60) Provisional application No. 61/063,735, filed on Feb. 6, 2008.

(51) Int. Cl. A61K 9/52; A61K 35/74; A61K 9/28; A61K 9/22; A61K 9/48; A61P 37/00; A61P 11/00

(52) U.S. Cl. 424/457; 424/93.4; 424/93.44; 424/93.45; 424/93.46; 424/93.3; 424/474; 424/468; 424/463

(57) ABSTRACT

Disclosed herein are compositions for treating a respiratory condition, preferably by enhancing immune response in a mammal, the compositions including a therapeutic amount of a probiotic strain of bacteria and a therapeutic amount of an additional component. Also included are methods of treating a respiratory condition, preferably by enhancing immune response, in a mammal. Kits containing the compositions, and instructions for applying the methods are also included. The method includes orally administering to the mammal a therapeutic amount of a probiotic strain of bacteria and a therapeutic amount of an additional component.

### CROSS REFERENCE TO RELATED APPLICATIONS

This application claims the benefit of U.S. Provisional Application No. 61/063,735, filed Feb. 6, 2008.

### FIELD OF THE INVENTION

The present invention relates to compositions and methods for treating a respiratory condition, preferably by enhancing immune response to a respiratory condition. More particularly, the present invention relates to compositions comprising a therapeutic amount of a probiotic strain of bacteria and a therapeutic amount of an additional component, and methods of using such compositions. Most particularly, the present invention relates to compositions and methods of using a probiotic strain of bacteria for treating a respiratory condition, preferably by enhancing immune response to a respiratory condition.

### BACKGROUND OF THE INVENTION

According to the Centers for Disease Control (CDC), in 2006 an estimated 56% of students aged 5-17 years missed between 1 and 5 days of school due to illness or injury, 10% missed 6 to 10 days, and 5% missed 11 or more days. In addition, it is estimated that 189 million school days are missed annually (an average of nearly 1 day per episode) due to a cold. In addition to children missing school, when children miss school, as a consequence often a parent misses work to stay home and care for the child. Often one or both parents and any siblings also contract the cold from the sick child which results in the siblings missing school and the parent(s) missing additional days of work either due to their own illness or caring for the sick child(ren).

However, colds are only one type of respiratory condition affecting the population, and respiratory conditions can be or be triggered by any of a variety of sources including allergens and/or pathogens of viral, bacterial or fungal origin. Common respiratory conditions include cold, influenza (flu), respiratory allergies, and asthma. Symptoms of respiratory conditions typically include coughing, sneezing, headaches, congestion, sore throat, stuffy nose, runny nose, fever, and the like.

Respiratory product types commonly used to treat such symptoms can generally be categorized as liquid elixirs, cough syrups, cold and flu capsules, cold and flu tablets, allergy tablets, effervescent tablets, mouth and nasal sprays, cough drops, and the like.

The most commonly employed products for treating respiratory conditions are ingested or bucally administered to inhibit and/or treat onset or fully developed respiratory symptoms. The products typically contain one or more actives dissolved or dispersed in a carrier system for ingestion or bucal delivery into the bloodstream. Although many consumers prefer respiratory products in the form of cough drops, liquids, or capsules, respiratory products in the form of powders and effervescent tablets have also met consumer needs in combating respiratory conditions.

Some compositions and methods for the prevention and treatment of the common cold and other respiratory conditions include treatments using combinations of anti-viral and anti-inflammatory compounds, treatments using orally administered aminocarboxylic acid compounds; treatment of cough and colds using compositions comprising non-steroidal anti-inflammatory drugs such as NSAIDS with antihistaminically effective materials such as chlorpheniramine; use of ionic zinc such that the ionic zinc contacts the nasal membrane; and use of probiotic strains of bacterial microorganisms which have been shown to have immunomodulatory effects, for example for the treatment of allergies.

Many different viruses and strains thereof can result in respiratory conditions and symptoms associated with respiratory conditions. The common cold, for example, is a complex syndrome that may be caused by any of over 200 antigenic viruses, among the most important of which is rhinovirus. Although distinct from the cold viruses, influenza viruses can and do produce many of the same symptoms.

Additionally, allergies are a particularly bothersome respiratory condition. Allergies can be, without limitation by theory, the result of hyper-reactivity of the immune system to foreign or self antigens. Type I allergy, such as allergic rhinitis (e.g., hay fever) or atopic dermatitis, occurs in allergic subjects upon exposure to environmental allergens (e.g., pollens or dust mites), and results in key clinical symptoms, similar to those of cold and flu, such as sore throat, cough, fatigue, sneeze, running nose, nasal drip, stuffy nose, nasal congestion, excessive mucus, sinus pressure, plugged ears, itchy nose, itchy, red, puffy, swollen, irritated and watery eyes.

In healthy status, the immune system maintains a balance between cytokines produced by different helper T lymphocyte subsets: Th1 and Th2 lymphocytes. In contrast, an allergic subject demonstrates a biased dysfunction of Th2 over Th1 that leads to an elevated IgE antibody production. The elevated production of IgE may be induced by hyper-reactivity of Th2 lymphocytes that secrete cytokines (e.g., IL-4, IL-5). Th1 cytokines (e.g., interferon-gamma, IL-12) may counteract Th2 cytokines and regain a healthy state in murine systems. IgE antibody-bound mast cells interact with allergen, triggering release of chemical mediators (e.g., histamine, leukotriene) and cause vasodilation and hypersecretion in various tissues. Antihistamines or leukotriene antagonists compete with the secreted inflammatory mediators from mast cells and significantly reduce clinical respiratory symptoms. Probiotic strains of bacteria have also been shown to have immunomodulatory effects when used in the treatment of allergies.

Thus, pinpointing specific causes of respiratory conditions can be difficult because there are a number of factors involved in the manifestation of respiratory conditions that are not fully understood. However, it is generally understood that factors including lowered immunity, stress, lack of sleep, too much exercise, malnutrition, seasonal changes, social activities, age, environmental toxins and even certain medications (i.e. immunosuppressive medications) can increase the risk of, and make individuals more susceptible to, various respiratory conditions.

Therefore, there remains a need for compositions and methods for enhancing immune response to a respiratory condition, in mammals, including human children. This need is particularly apparent with respect to children because the use of common cold/flu actives for children 12 years of age and under has recently come into question with respect to both efficacy and safety of those actives in children. Therefore, there is an unmet need for compositions and methods for enhancing immune response, including reducing susceptibility to respiratory conditions, preventing and treating respiratory conditions, and reducing the severity and duration of respiratory conditions, which are safe, effective, palatable and easy to administer and use.

### SUMMARY OF THE INVENTION

The present invention comprises compositions for treating a respiratory condition, preferably by enhancing immune response to a respiratory condition in a mammal, comprising a therapeutic amount of a probiotic strain of bacteria and a therapeutic amount of an additional component. The present invention also includes methods of treating a respiratory condition, preferably by enhancing immune response to a respiratory condition in a mammal, comprising orally administering to the mammal a therapeutic amount of a strain of Lactobacillus and a therapeutic amount of an additional component. The present invention also comprises kits containing the compositions. The present compositions and methods can also or alternatively include a strain of Bifidobacterium and/or other additional components. The compositions of the present invention can formed as a single composition or separate compositions packaged together in a kit.

### DETAILED DESCRIPTION OF THE INVENTION

Various documents including, for example, publications and patents, are recited throughout this disclosure. All documents are hereby incorporated by reference.

Referenced herein may be trade names for components including various ingredients utilized in the present invention. The inventors herein do not intend to be limited by materials under a certain trade name. Equivalent materials (e.g., those obtained from a different source under a different name or reference number) to those referenced by trade name may be substituted and utilized in the descriptions herein.

In the description of the invention various embodiments and/or individual components are disclosed. As will be apparent to the ordinarily skilled practitioner, all combinations of such embodiments and components taught in the disclosure are possible and can result in preferred executions of the present invention.

All percentages and ratios are calculated by weight unless otherwise indicated. All percentages, parts and ratios are calculated based on the total composition unless otherwise indicated. Weights as they pertain to listed ingredients are based on the specific ingredient level and, therefore, do not include carriers or by-products that may be included in commercially available materials, unless otherwise specified.

As used herein, the terms “mixture” and “combination” include multiple components or ingredients formed into one resulting component, components that can be separate but contained in a single dosage form, and components that can be administered in the same treatment regimen even if not physically formed into a single component or contained in a single dosage form. As used herein, the terms “mixture” and “combination” may be used interchangeably.

As used herein, “mammal” includes but is not limited to humans as well as domestic animals including cat, dog, cow, rabbit, and horse.

As used herein, “respiratory condition” includes susceptibility to, risk of, and onset of symptoms of the conditions described herein. “Respiratory condition” as used herein refers to conditions including, but not limited to, respiratory tract viral infections, respiratory tract bacterial infections, respiratory tract fungal infections, allergies (for example to pollen, fungi, and environmental allergens), asthma, auto-immune conditions, rhinitis, sinusitis, bronchiolitis, acute respiratory distress syndrome (ARDS), severe acute respiratory syndrome (SARS), respiratory cancer, emphysema, COPD, difficulty breathing, cough, and conditions pursuant to respiratory surgeries (including pre- and post-operative management).

As used herein, “immune response” includes all of the specific and non-specific processes and mechanisms involved in how the body defends and repairs itself against bacteria, viruses, fungi, allergens and all substances, insults, challenges, biological and/or physical invasions of the body that are harmful to the body.

As used herein “enhancing immune response” means a change to the immune system which provides a benefit to the mammal. “Enhancing” the immune response also includes prevention, treatment, cure, mitigation amelioration, inhibition and/or alleviation of a respiratory condition and/or symptoms thereof. “Enhancing the immune system” results in benefits including improved quality of life; improved mood and/or reduced stress; improved concentration; better overall health; improved respiratory health; preserving, maintaining and/or restoring normal ability to perform normal daily tasks, including the ability to go to work and/or school; providing, supporting and/or maintaining normal vitality and energy levels; and enhancing sleep including quality of sleep. “Enhancing the immune system” also includes maintaining, supporting and/or strengthening natural defenses, and enhancing wellness and overall immune system health.

As used herein “cold, influenza and allergy-like symptoms” refers to symptoms typically associated with respiratory conditions as defined herein. These symptoms include, but are not limited to, nasal congestion, chest congestion, sneezing, rhinorrhea, fatigue, malaise, cough, fever, chills, body ache, sore throat, headache, excessive mucus, sinus pressure, nasal drip, runny nose, itchy eyes, watery eyes and other known cold, influenza and allergy-like symptoms.

As used herein “respiratory viruses” refers to those viruses that are causal agents of respiratory conditions that result in cold and influenza-like symptoms. Non-limiting examples of such viruses include Rhinovirus, Myxovirus (Influenza virus), Paramyxovirus (Parainfluenza virus), Respiratory Syncytial virus, Adenovirus and Coronavirus.

As used herein “respiratory bacteria” refers to those bacteria that are causal agents of respiratory conditions that result in cold and influenza-like symptoms. Non-limiting examples of such bacteria include Hemophilus influenzae, mycobacteria, pasteurella, Pneumocystis jiroveci, Mycobacterium tuberculosis, Streptococcus pheumoniae, bacteria pneumonia and Klebsiella pneumoniae.

As used herein “respiratory fungi” refers to those fungi that are causal agents of respiratory conditions that result in cold and influenza-like symptoms. Non-limiting examples of respiratory fungi and fungally caused respiratory conditions include aspergillosis, hisoplasmosis, Blastomyces, dermatitidis, Cryptococcus neoformas, Coccidioidomycosis, and Pneumocystis jiroeci.

As used herein, a “probiotic” microorganism or strain of microorganism confers beneficial functions and/or effects to a host animal when a “probiotic” microorganism is administered to a host animal at a therapeutically effective amount. As used herein “probiotic” microorganism includes bacteria, bacterial homogenates, ground bacterial cells, bacterial proteins, bacterial extracts, bacterial ferment supernatants, and mixtures thereof “Probiotic” microorganisms also include natural and/or genetically modified microorganisms, viable or dead; processed compositions of microorganisms; their constituents and components such as proteins and carbohydrates, extracts, distillates, isolates, purified fractions, and mixtures thereof of bacterial ferments that beneficially affect a host. Although a use of probiotic microorganisms herein can be in the form of viable cells, use can be extended to non-viable cells such as killed cultures, or compositions containing beneficial factors expressed by the probiotic microorganism. Killed cultures may include thermally killed microorganisms, or microorganisms killed by exposure to altered pH or subjected to pressure. “Probiotic” microorganism is further intended to include metabolites generated by the microorganisms during fermentation, if such metabolites are not separately indicated. These metabolites may be released to the medium of fermentation, or they may be stored within the microorganism.

The abbreviation CFU (referring to “colony-forming unit”) as used herein designates the number of bacterial cells revealed by microbiological counts on agar plates, as will be commonly understood in the art.

The term “pharmaceutically acceptable carrier” refers to any solid, liquid or gas combined with components of the compositions of the present invention to deliver the components to the user. These vehicles are generally regarded as safe for use in humans, and are also known as carriers or carrier systems.

The term “therapeutic amount” of a component, composition, or like material as used herein refers to a concentration or amount of any active defined herein that is ingested, including ingestion by buccal administration, that is effective to provide the desired effect or benefit to a host animal without undue adverse side effects (such as toxicity, irritation, or allergic response), commensurate with a reasonable benefit/risk ratio when used in the manner of this invention. In the present invention, desired effects and/or benefits include enhancement of the immune system including treatment, prevention or resistance of respiratory conditions in a mammal. The specific “therapeutic amount” will, obviously, vary with such factors as the particular condition being treated, the particular composition to be used, the physical condition of the treated mammal, the size and weight of the treated mammal, the duration of treatment, the nature of concurrent therapy (if any), the specific dosage form to be used, other components present in a given dosed composition, and the dosage regimen desired for the component or composition.

Additional definitions are provided as necessary herein as they occur.

The compositions and methods of the present invention can comprise, consist of, or consist essentially of the elements and limitations of the invention described herein, as well as any of the additional or optional ingredients, components, limitations, or steps described herein.

### Compositions

The present invention comprises compositions comprising a therapeutic amount of a probiotic strain of bacteria and a therapeutic amount of an additional component, and methods of using the compositions, in mammals, as defined herein. The present invention also can comprise kits containing the compositions, with the compositions formed as single compositions or as separate compositions packaged together in a kit.

In one embodiment, the probiotic strain of bacteria herein is able to maintain viability following transit through the gastrointestinal tract. This is desirable in order for live cultures of the bacteria to be taken orally, and for colonization to occur in the intestines and bowel following transit through the esophagus and stomach. Colonization of the intestine and bowel by the probiotic strain of bacteria is desirable for long term probiotic benefits to be delivered to the host. Oral administration of non-viable cells or purified isolates thereof can induce temporary benefits. However, if the bacteria are not viable, they are not able to grow, and are more limited in ability to continuously deliver a probiotic effect. As a result, this may require the host to be dosed regularly in order to maintain the health benefits. In contrast, viable cells that are able to survive gastric transit in viable form, and subsequently colonize by adhering to and proliferating on the gut mucosa, are better able to deliver probiotic effects continuously.

The compositions utilized in the compositions and methods herein comprise a probiotic strain of bacteria. Non-limiting examples of bacteria suitable for use herein include strains of Streptococcus lactis, Streptococcus cremoris, Streptococcus diacetylactis, Streptococcus thermophilus, Lactobacillus bulgaricus, Lactobacillus acidophilus, Lactobacillus helveticus, Lactobacillus bifidus, Lactobacillus casei, Lactobacillus lactis, Lactobacillus plantarum, Lactobacillus rhamnosus, Lactobacillus delbruekii, Lactobacillus thermophilus, Lactobacillus fermentii, Lactobacillus salivarius, Lactobacillus reuteri, Lactobacillus brevis, Lactobacillus paracasei, Lactobacillus gasseri, Pediococcus cerevisiae, Bifidobacterium longum, Bifidobacterium infantis, Bifidobacterium adolescentis, Bifidobacterium bifidum, Bifidobacterium animalis, Bifidobacterium pseudolongum, Bifidobacterium thermophilum, Bifidobacterium lactis, Bifidobacterium bulgaricus, Bifidobacterium breve, Bifidobacterium subtilis, Escherichia coli and strains of the genera including Bacillus, Bacteroides, Enterococcus (e.g., Enterococcus faecium) and Leuconostoc, and mixtures and/or combinations thereof.

Embodiments of the compositions and methods of the present invention comprise strains of lactic acid bacteria selected from the genera Lactobacillus and Bifidobacterium, such as Lactobacillus acidophilus, Lactobacillus fermentum and Bifidobacterium lactis, and combinations and/or mixtures thereof. In one embodiment, the methods herein comprise administration of a composition comprising a therapeutic amount of the lactic acid bacteria.

Non-limiting examples of Lactobacillus suitable for use herein include strains of Lactobacillus bulgaricus, Lactobacillus acidophilus, Lactobacillus helveticus, Lactobacillus bifidus, Lactobacillus casei, Lactobacillus lactis, Lactobacillus plantarum, Lactobacillus rhamnosus, Lactobacillus delbruekii, Lactobacillus thermophilus, Lactobacillus fermentii, Lactobacillus salivarius, Lactobacillus reuteri, Lactobacillus brevis, Lactobacillus paracasei, Lactobacillus gasseri, and combinations thereof.

A non-limiting example of a Lactobacillus strain suitable for use herein includes the Lactobacillus acidophilus strain identified as LAFTI® L10 deposited under accession number CBS 116411 available from DSM Corporation based in the Netherlands.

In one embodiment herein, the compositions can comprise at least about 10⁴ CFU of Lactobacillus, alternatively from about 10⁴ to about 10¹⁴ CFU of Lactobacillus, in another embodiment from about 10⁶ to about 10¹² CFU of Lactobacillus, in another embodiment from about from about 10⁸ to about 10¹¹ CFU of Lactobacillus, per unit dose of the composition.

Other non-limiting examples of a Lactobacillus strains suitable for use herein include the Lactobacillus acidophilus strain identified as CL-92 deposited in Japan at International Patent Organism Depository, FERM BP-4981, the Lactobacillus acidophilus strain identified as CL0062 deposited in Japan at International Patent Organism Depository, FERM BP4980, and the Lactobacillus fermentum strain identified as CP34 and deposited in Japan at International Patent Organism Depository, FERM BP-8383. These organisms, have been shown, as described in US Patent Application Publication Number US 2005/0214270, to provide anti-allergic effects by suppressing IgE levels in mice, and by reducing allergy symptoms and decreasing IgE titer in the blood in humans.

In one embodiment, the compositions can comprise at least about 1×10⁴, alternatively at least about 1×10⁹, alternatively at least about 1×10¹⁰, and alternatively at least about 5×10¹⁰ cells per day of the probiotic strain of bacteria, which can be administered in a single dose, or in a plurality of doses.

In one embodiment, the methods herein comprise administration of a composition comprising a therapeutic amount a strain of Bifidobacterium, which can be mammalian, in addition to, or in alternative to, a Lactobacillus as described herein. The mammal treated and a mammalian source of Bifidobacterium isolation may be, but need not be, independent.

Non-limiting examples of Bifidobacterium suitable for use herein include strains of Bifidobacterium longum, Bifidobacterium infantis, Bifidobacterium adolescentis, Bifidobacterium bifidum, Bifidobacterium animalis, Bifidobacterium pseudolongum, Bifidobacterium thermophilum, Bifidobacterium lactis, Bifidobacterium bulgaricus, Bifidobacterium breve, Bifidobacterium subtilis, and mixtures and/or combinations thereof.

A non-limiting example of a Bifidobacterium strain suitable for use herein includes Bifidobacterium lactis identified as LAFTI® 94 deposited under accession number CBS 118529 that can be purchased from DSM Corporation. Other examples of useful Bifidobacterium strains include Bifidobacterium longum strain identified as BB-536 (Morinaga & Co., LTD, Japan)

In one embodiment herein, the compositions used in the methods herein comprise at least about 10⁴ CFU of Bifidobacterium, alternatively from about 10⁴ to about 10¹⁴ CFU of Bifidobacterium, in another embodiment from about 10⁶ to about 10¹² CFU of Bifidobacterium, in another embodiment from about from about 10⁸ to about 10¹¹ CFU of the Bifidobacterium, per unit dose of the composition.

In one embodiment, the probiotic strain of bacteria, can comprise a freeze-dried powder (as would be understood by one of skill in the art) can comprise from about 1% to about 50%, alternatively from about 1% to about 40%, alternatively from about 1% to about 30%, and alternatively from about 2% to about 20%, by weight of the composition.

### Additional Components

The compositions of the present invention can optionally comprise one or more additional components. By way of non-limiting example, an additional probiotic strain of bacteria; one or more of prebiotics and/or fiber; vitamins; minerals, metals and elements; plant-derived components; fungal-derived components; carotenoids; anti-oxidants; and mixtures/combinations thereof can be used.

The compositions of the present invention can comprise, by way of non-limiting example, one or more probiotic strains of bacteria plus one or more of an additional probiotic strain of bacteria, a prebiotic, a fiber, vitamins, minerals, elements, plant-derived components, fungal-derived components, carotenoids, and antioxidants. Non-limiting examples of some additional components are provided below.

### Prebiotics/Fiber

The compositions of the present invention comprising the probiotic used herein can comprise a prebiotic and/or a fiber.

As used herein, the term “prebiotic” includes substances or compounds that beneficially affect the host mammal by selectively promoting the growth and/or activity of one or more probiotic bacteria in the gastro-intestinal tract of the host mammal, thus maintaining normal health or improving health of the host. Typically, prebiotics are carbohydrates, (such as oligosaccharides), but the term “prebiotic” as used herein does not preclude non-carbohydrates. Many forms of “fiber” exhibit some level of prebiotic effect. Thus, there is considerable overlap between substances that can be classified as “prebiotics” and those that can be classified as “fibers”.

Non-limiting examples of prebiotics suitable for use in the compositions and methods include psyllium, fructo-oligosaccharides, inulin, oligofructose, galacto-oligosaccharides, isomalto-oligosaccharides xylo-oligosaccharides, soy-oligosaccharides, gluco-oligosaccharides, mannan-oligosaccharides, arabinogalactan, arabinxylan, lactosucrose, gluconannan, lactulose, polydextrose, oligodextran, gentioligosaccharide, pectic oligosaccharide, xanthan gum, gum arabic, hemicellulose, resistant starch and its derivatives, and mixtures and/or combinations thereof.

When present, the compositions can comprise from about 100 mg to about 100 g, alternatively from about 500 mg to about 50 g, and alternatively from about 1 g to about 40 g, of prebiotic, per daily dose of the composition.

### Fiber

As used herein, the term “fiber” means carbohydrate polymers including those naturally occurring in food as consumed, those having been obtained from food raw material by physical, enzymatic or chemical means, and synthetic carbohydrate polymers, which are resistant to digestion and absorption in the small intestine and have partial fermentation in the large intestine.

Non-limiting examples of fiber and analogous carbohydrate polymers suitable for use in the compositions and methods of the present invention include pectins, psyllium, guar gum, xanthan gum, alginates, gum arabic, fructo-oligosaccharides, inulin, agar, beta-glucans, chitins, dextrins, lignin, celluloses, non-starch polysaccharides, carrageenan, and mixtures and/or combinations thereof.

In one embodiment, the fiber is glucose polymers, preferably those which have branched chains. Among such suitable fibers is one marketed under the tradename “Fibersol2”, commercially available from Matsutani Chemical Industry Co., Itami City, Hyogo, Japan.

Other non-limiting examples of suitable fibers include oligosaccharides, such as inulin and its hydrolysis products commonly known as fructo-oligosaccharides, galacto-oligosaccharides, xylo-oligosaccharides, and oligo derivatives of starch.

The fiber can be provided in any suitable form. A non-limiting example is in the form of a plant material which contains the fiber. Non-limiting examples of suitable plant materials include asparagus, artichoke, onion, wheat, chicory, beet pulp, residues of these plant materials, and mixtures thereof.

A non-limiting example of a fiber from such a plant material is inulin extract from extract of chicory. Suitable inulin extracts can be obtained from Orafti SA of Belgium under the trademark Raftiline®. Alternatively the fiber can be in the form of a fructo-oligosaccharide which can be obtained from Orafti SA of Belgium under the trademark Raftilose®. Alternatively, an oligo-saccharide can be obtained by hydrolyzing inulin, by enzymatic methods, or by using microorganisms as will be understood by those of skill in the art. Alternatively the fiber can be Inulin and/or de-sugared inulin available from Cargill Health & Food Technologies, Wayzata, Minn., USA, or from Cosucra SA, Warcoing, Belgium.

When present, the compositions can comprise from about 100 mg to about 100 g, alternatively from about 500 mg to about 50 g, alternatively from about 1 g to about 40 g, of fiber, per daily dose of the composition.

### Vitamins

The compositions of the present invention can optionally comprise one or more vitamins. When certain vitamins, minerals, metals, elements and the like are included as additional components in capsule, tablet and powder forms, the actual amounts of these many of these components, in grams per unit dose, are often extremely small, and make the individual components difficult to handle, measure and process. Therefore such components are commonly prepared or purchased as a premix in or on a carrier such as sucrose or lactose. The total amount of vitamin(s), by weight, if provided in a premix on or in a suitable carrier, can comprise from about 1% to about 50%, alternatively from about 1% to about 40%, and alternatively from about 2% to about 30%, by weight of the composition. Therefore, when vitamins, minerals, metals and elements are exemplified herein as a % by weight of a composition, such weight % includes any carrier present.

With respect to the weight percent of a given vitamin as a percent of a premix or vitamin-carrier mix, such percentages can vary greatly depending on the vitamin and the amount of vitamin desired, as would be understood by one of skill in the art. Generally, however, for vitamins in or on a carrier, the vitamin can comprise, as a weight percent of vitamin to carrier, from about 0.0001% to about 50%, alternatively from about 0.001% to about 45%, alternatively from about 0.001% to about 40%, by weight of the vitamin-carrier composition.

### Vitamin D

The compositions of the present invention can comprise Vitamin D. Non-limiting examples of vitamin D suitable for use in the present invention include vitamin D₃ (cholecalciferol), vitamin D₂ (ergocalciferol) and combinations thereof. Additional non-limiting examples include metabolites of vitamin D including calcidiol, calcitriol and combinations thereof. The vitamin D can be derived from natural or synthetic sources, including from an extract of solanum glaucophylum (malacoxylon), trisetum flavescens (goldhafer) or cestrum diurnum. Both the pure vitamin D and/or glycosides of the vitamin D can be used.

Vitamin D is a unique nutrient in that its principal source is not the diet, but via synthesis in the skin upon exposure to UV light, typically sunlight in the summer months. In the skin, 7-dehydrocholesterol, derived from cholesterol, is converted by the action of UV light into Previtamin D₃ (precalciferol), which then undergoes a thermal conversion to Vitamin D₃ (cholecalciferol). Whether the cholecalciferol is synthesized in the skin or absorbed through the gut, it is transported to the liver where it is converted to 25-OH cholecalciferol (calcidiol) (25-hydroxycholecalciferol). This is the form that is ordinarily assayed in the blood. Calcidiol is eventually transported to the kidneys, where it is converted to 1,25-(OH)₂ cholecalciferol (calcitriol) the active form.

In order to provide meaningful symptom relief during a respiratory condition specific blood levels of 25-hydroxycholecalciferol need to be achieved. The compositions of the present invention can comprise from about 50 IU to about 500,000 IU, alternatively from about 500 IU to about 500,000 IU, alternatively from about 1,000 IU to about 500,000 IU of cholecalciferol, per daily dose, alternatively from about 2,000 IU to about 100,000 IU, alternatively from about 10,000 IU to about 50,000 IU, and alternatively from about 20,000 IU to about 40,000 IU, per daily dose, of cholecalciferol. With these levels of cholecalciferol administered to the human, the specific increase in blood levels of the 25-hydroxycholecalciferol in the human user will be from about 1 ng/ml to about 40 ng/ml, alternatively from about 2 ng/ml to about 30 ng/ml, alternatively from about 4 ng/ml to about 20 ng/ml, as determined by the methodology calcidiol 25-Hydroxyvitamin D ¹²⁵I RIA Kit radioimmunoassay (RIA) Catalog No./REF./KAT.-NR.:68100E manufactured, distributed and available from DiaSorin Inc., Stillwater, Minn. USA 55082.

To treat the symptoms of a respiratory condition that is already underway, the mammal, for example a human, can be administered a composition comprising in a single dose form, or multiple dose form, from about 50 IU to about 500,000 IU, alternatively from about 500 IU to about 500,000 IU, alternatively from about 1000 IU to about 500,000 IU, alternatively from about 5,000 IU to about 500,000 IU, alternatively from about 10,000 IU to about 100,000 IU, and alternatively from about 20,000 to about 50,000 IU of cholecalciferol per day.

To treat or prevent the symptoms of a respiratory condition, the mammal can be administered the composition comprising, in a single dose form, or multiple dose form, from about 50 IU to about 10,000 IU, alternatively from about 500 IU to about 10,000 IU, alternatively from about 1,000 IU to about 5,000 IU, alternatively from about 2,000 IU to about 5,000 IU, and alternatively from about 2,000 IU to about 4,000 IU of cholecalciferol per day.

The compositions can also comprise Vitamin D₂ (ergocalciferol). The compositions can comprise from about 50 IU to about 500,000 IU, alternatively from about 500 IU to about 500,000 IU, alternatively from about 1,000 IU to about 500,000 IU, and, alternatively from about 5,000 IU to about 500,000 IU of Vitamin D₂, per daily dose of the composition.

When present, the compositions can comprise from about 1.25 μg to about 12.5 mg, alternatively from about 12.5 μg to about 12.5 mg, alternatively from about 25 μg to about 12.5 mg, and alternatively from about 125 μg to about 12.5 mg of vitamin D₃ and/or D₂, per daily dose of the composition.

### Vitamin C

The compositions of the present invention can also comprise Vitamin C. It is believed that over 20% of subjects with colds have suboptimal levels of Vitamin C. The preferred form of Vitamin C for use in the composition is as ascorbic acid or the equivalent of a salt of ascorbic acid or the equivalent of a derivative of ascorbic acid. The vitamin C may either be in an immediate release form or a sustained release form.

Vitamin C (as calcium ascorbate) is a water-soluble compound, found in aqueous cellular compartments and is a line of defense against direct free radical exposure. Vitamin C maintains oxidative balance by effectively scavenging free radicals produced in the aqueous cellular cytoplasm and by recycling (protecting) vitamin E in cellular membranes. A preferred form of Vitamin C is as calcium ascorbate. Without being limited by theory, it is believed that ascorbate enhances the antioxidant action of vitamin E by reducing reduction of the tocopheroxyl radical. The reactions between the tocopheroxyl radical and ascorbate provide a mechanism for exporting oxidative free radicals away from the cellular membranes. In essence, tocopherols protect membranes by stopping propagation reactions of lipid peroxy radicals and ascorbate acts by protecting the membrane against possible damage from the tocopheroxyl radical. Thus, ascorbate helps to maintain oxidative balance by scavenging free radicals and recycling the useful forms of other antioxidants, such as vitamin E.

When present, the compositions can comprise from about 20 mg to about 2000 mg, alternatively from about 80 mg to about 1500 mg, and alternatively from about 100 mg to about 1000 mg of Vitamin C, per daily dose of the composition.

### Vitamin A

The compositions of the present invention can also comprise Vitamin A and/or carotene. Vitamin A and carotene can be obtained from either animal or vegetable sources. The animal form is divided between retinol and dehydroretinol whereas the vegetable carotene can be split into four very potent groups—alpha-carotene, beta-carotene, gamma-carotene and crypto-carotene. Vitamin A assists the immune system, and because of its antioxidant properties protects against disease.

Non-limiting examples of the Vitamin A useful in the present invention include vitamin A, retinol, retinyl palmitate, retinyl acetate, retinyl proprionate, beta-carotene, alpha carotene, beta-cryptoxanthin, and mixtures thereof.

When Vitamin A or one of the forms of Vitamin A is present, the compositions comprise from about 100 IU to about 10,000 IU, alternatively from about 300 IU to about 5,000 IU, alternatively from about 400 IU to about 2,000 IU, and alternatively from about 500 IU to about 1,000 IU of Vitamin A and/or form thereof, per day of the composition. The amount of Vitamin A species may be expressed as IU or as RAE (Retinol Activity Equivalent), which is equal to an equivalent amount of retinol in micrograms. For example, 10,000 IU Vitamin A is equivalent to 3000 RAE or 3000 μg retinol.

When Vitamin A (retinol) is present, the compositions can comprise from about 30 μg to about 4545 μg, alternatively from about 90 μg to about 1500 μg, alternatively from about 120 μg to about 600 μg, and alternatively from about 150 μg to about 300 μg of Vitamin A (retinol), per daily dose of the composition.

### B Vitamins

The compositions of the present invention can comprise one or more B Vitamins. The B Vitamins are water-soluble vitamins that play important roles in cell metabolism. B Vitamins are a collection of chemically distinct vitamins that often coexist in the same foods. Supplements containing eight specific B Vitamins are generally referred to as a “Vitamin B complex”. Individual B Vitamin supplements are referred to by the specific name of each vitamin (e.g. B₁, B₂, B₃, etc). The B Vitamins often work together to deliver a number of health benefits and these include, but not limited to maintenance and support of metabolic rate, maintenance of healthy skin and muscle tone, enhance immune and nervous system function, promote cell growth and division and together can also help combat the symptoms of stress, depression, and cardiovascular disease. The B Vitamin most associated with immune function is Vitamin B6 as deficiencies in this vitamin have an effect on both cellular and humoral immunity. In addition, it has been shown that B Vitamins are an essential part of energy production and can improve mood. Mood improvement, in turn, has been associated with lower incidence of colds and reduced respiratory symptoms.

All B Vitamins are water soluble, and are dispersed throughout the body. Most of the B Vitamins must be replenished daily, since any excess is excreted in the urine.

Non-limiting examples of Vitamin B useful in the present invention include vitamin B1 (thiamin), Vitamin B2 (Riboflavin), Vitamin B3 (niacin), Vitamin B5 (pantothenic acid), Vitamin B6 (pyridoxine, pyridoxal, or pyridoxamine), Vitamin B7 (Biotin), Vitamin B9 (Folic acid), Vitamin B12 (cyanobalmin), and mixtures thereof.

When Vitamin B1 is present, the compositions can comprise from about 200 ug to about 50 mg, alternatively from about 400 μg to about 20 mg, and alternatively from about 500 μg to about 10 mg of Vitamin B1, per daily dose of the composition.

When Vitamin B2 is present, the compositions can comprise from about 100 μg to about 200 mg, alternatively from about 200 μg to about 100 mg, and alternatively from about 500 μg to about 50 mg of Vitamin B2, per daily dose of the composition.

When Vitamin B3 is present, the compositions can comprise from about 1 mg to about 500 mg, alternatively from about 2 mg to about 250 mg, and alternatively from about 5 mg to about 100 mg of Vitamin B3, per daily dose of the composition.

When Vitamin B5 is present, the compositions can comprise from about 500 μg to about 1000 mg, alternatively from about 1000 μg to about 500 mg, and alternatively from about 2000 μg to about 100 mg of Vitamin B5, per daily dose of the composition.

When Vitamin B6 is present, the compositions can comprise from about 200 μg to about 500 mg, alternatively from about 500 μg to about 250 mg, and alternatively from about 1000 μg to about 100 mg of Vitamin B6, per daily dose of the composition.

When Vitamin B7 is present, the compositions can comprise from about 1 μg to about 200 μg, alternatively from about 2 μg to about 100 μg, and alternatively from about 5 μg to about 50 μg of Vitamin B7, per daily dose of the composition.

When vitamin B9 is present, the compositions can comprise from about 50 μg to about 2000 μg, alternatively from about 100 μg to about 1000 μg, and alternatively from about 200 μg to about 500 μg of Vitamin B9, per daily dose of the composition.

When vitamin B12 is present, the compositions can comprise from about 0.5 μg to about 3000 μg, alternatively from about 1 μg to about 1500 μg, and alternatively from about 2 μg to about 750 μg of Vitamin B12, per daily dose of the composition.

### Minerals, Metals, and Elements

The compositions of the present invention can include minerals, metals and elements. Non-limiting examples of minerals, metals, and elements useful in the compositions of the present invention include: iron, iodine, zinc, copper and selenium. Adequate intake of iron, zinc, copper and selenium support a Th1 cytokine-mediated immune response which helps circumvent an anti-inflammatory Th2 response and an increased risk of extracellular infections. When present, the minerals, metals and/or elements, on or in a suitable carrier, comprise from about 1% to about 50% by weight of the composition and alternatively from about 2% to about 30%, by weight of the composition.

### Iron

The compositions of the present invention can comprise iron. Iron (as Fe²⁺, ferrous ion) is a necessary trace element used by almost all living organisms. It is used in hemoglobin which carries oxygen to the cells. Too little iron can cause anemia, resulting in fatigue and tiredness and has been associated with decreased cellular immunity. However, too much iron can be lethal.

Without being limited by theory, it is believed that the addition of iron to the composition of the present invention provides an energy and/or immunoregulatory or enhancing benefit.

A non-limiting example of iron suitable for use with the present invention is the bisclycinate salt form of iron, available under the tradename “Ferrochel” from Albion Laboratories Inc., Clearfield, Utah, USA.

When present, the compositions cancomprise from 2 mg to about 18 mg, alternatively from about 3 mg to about 15 mg, and alternatively from about 3 mg to about 10 mg of iron, per daily dose of the composition.

### Iodine

The compositions of the present invention can comprise iodine. Iodine is required in trace amounts in most living organisms, and is commonly used in medicine. Iodine's known role in biology is as a constituent of the thyroid hormones. Natural sources of iodine include sea life such as kelp and certain seafood. Iodine deficiency gives rise to hypothyroidism, symptoms of which are extreme fatigue, goitre, mental slowing, depression, weight gain, and low basal body temperatures. Iodine deficiency is also a cause of preventable mental retardation, an effect which happens primarily when babies and small children lack the element. The addition of iodine to table salt has largely eliminated this problem in the wealthier nations, but iodine deficiency remains a serious public health problem in the developing world. Thus, although only generally present and required in trace amounts, iodine has a key role in overall wellness, particularly in children.

When present, the compositions can comprise from about 20 μg to about 1 mg iodine, alternatively from about 30 μg to about 500 μg, and alternatively from about 30 μg to about 100 μg of iodine, per daily dose of the composition.

### Zinc

The compositions of the present invention can comprise zinc. Zinc is a trace element important to many biological and biochemical pathways. Zinc activates may enzymes such as those critical for antioxidant activity and protein/carbohydrate metabolism, metalloproteins, and zinc binds proteins to induce appropriate function, is secreted cy cells for initiation of biological responses, and directly modulates the host immune system. Zinc salts are effective against pathogens in direct application, and both zinc gluconate and zinc gluconate glycine have been shown to shorten the duration of symptoms of the common cold.

Without being limited by theory, it is believed that the addition of zinc to the composition of the present invention provides an improved benefit in the incidence, duration, and severity of respiratory conditions in addition to the enhancement of probiotic efficacy.

When present, the compositions can comprise zinc in an amount from about 1 mg to about 50 mg, alternatively from about 1 mg to about 30 mg, and alternatively from about 1 mg to about 25 mg of zinc, per daily dose of the composition.

### Copper

The compositions of the present invention can comprise copper. Copper is a trace element that binds cytochrome c oxidase and superoxide dismutase; and binds metalloenzymes involved in hemoglobin formation, drug metabolism, carbohydrate metabolism, collagen and elastin cross-linking, and the antioxidant defense mechanism. Additionally, copper is used for biological electron transport, wound healing, red blood cell production, and immune support and performance. Copper has been used as an anti-microbial and an anti-arthritic agent.

When present, the compositions can comprise from about 200 μg to 10 mg, alternatively from about 500 μg to about 9 mg, and alternatively from about 1 mg to about 9 mg of copper, per daily dose of the composition.

### Selenium

The compositions of the present invention can comprise selenium. Although it is toxic in large doses, selenium is an essential micronutrient for animals. In humans, selenium is a trace element nutrient which functions as a cofactor for reduction of antioxidant enzymes such as glutathione peroxidases and certain forms of thioredoxim reductase found in animals and some plants. Selenium may act as an antioxidant and/or enhance immune activity.

When present, the compositions can comprise from about 15 μg to about 400 mg, alternatively from about 20 μg to about 300 mg, and alternatively from about 50 μg to about 200 mg of selenium, per daily dose of the composition.

### Plant-Derived Components

The compositions of the present invention can comprise plant-derived components. As used herein plant-derived components include herbs including those used in traditional native American, Chinese, aruvedic and Japanese medicine, herbal extracts, and isolated active components of plants from the flower, leaves, stems, roots, and seeds of plants.

### Polyphenols

The compositions of the present invention can comprise at least one polyphenol. Non-limiting examples of sources of polyphenols useful in the present invention include tea extract, rosemary extract, rosemarinic acid, coffee extract, coffeic acid, turmeric extract, blueberry extract, grapeseed extract, and mixtures thereof. Polyphenols have antioxidant activity and anti-inflammatory effects.

The polyphenol utilized in accordance with the present invention can be administered to a mammal in a variety of forms adapted to a chosen route of administration, for example, orally, parenterally, intravenously, subcutaneously, and like routes. A preferred method of administration is oral administration.

When present, the compositions can comprise from about 0.01% to about 90%, alternatively from about 0.1% to about 35%, alternatively from about 1% to about 15%, alternatively from about 1% to about 10%, and alternatively from about 3% to about 10% of a polyphenol, by weight of the composition.

### Tea Extract

The compositions of the present invention can comprise tea extract. Tea extract contains polyphenols. Nonlimiting examples of extracts include extracts of Camellia sinensis. The tea extract has antioxidant activity so as to quench reactive oxygen species such as singlet oxygen, superoxide and hydroxyl radicals. Tea extract enhances the antioxidant defense system by preserving antioxidant enzyme activity, and can be useful to enhance immune response to a respiratory condition. Nonlimiting sources of tea extract for use in the present invention are black tea, white tea, oolong tea, and/or green tea.

When tea extract is present, the compositions can comprise from about 0.01% to about 90%, alternatively from about 0.1% to about 35%, alternatively from about 1% to about 15%, alternatively from about 1% to about 10%, and alternatively from about 3% to about 10% tea extract, by weight of the composition.

When tea extract is green tea, the compositions can comprise from about 0.01% to about 90%, alternatively from about 0.1% to about 35%, alternatively from about 1% to about 15%, alternatively from about 1% to about 10%, and alternatively from about 3% to about 10% green tea extract, by weight of the composition.

### Rosemary Extract

The compositions of the present invention can comprise rosemary extract. Rosemary extract is a polyphenol. Constituents of rosemary or rosemary extract are coffeic acid and its derivatives such as rosemarinic acid. These compounds have antioxidant activity and anti-inflammatory effects which can be beneficial in enhancing immune response to a respiratory condition. Non-limiting sources of rosemary extract suitable for use in the present invention include rosemary.

When rosemary extract is present, the compositions can comprise from about 0.01 to about 90%, alternatively from about 0.1% to about 35%, alternatively from about 1% to about 15%, alternatively from about 1% to about 10%, and alternatively from about 3% to about 10% rosemary extract, by weight of the composition.

When rosemarinic acid is present, the compositions can comprise from about 0.01% to about 90%, alternatively from about 0.1% to about 35%, alternatively from about 1% to about 15%, alternatively from about 1% to about 10%, and alternatively from about 3% to about 10% rosemarinic acid, by weight of the composition.

### Coffee Extract

The compositions of the present invention can comprise coffee extract. Coffee extract is a polyphenol. The main constituent of coffee extract is coffeic acid and is, without being limited by theory, believed to display antioxidant activity which can be useful in enhancing immune response to a respiratory condition.

When coffee extract present, the compositions can comprise from about 0.01% to about 90%, alternatively from about 0.1% to about 35%, alternatively from about 1% to about 15%, alternatively from about 1% to about 10%, and alternatively from about 3% to about 10% coffee extract, by weight of the composition.

When coffee extract is present non-limiting sources of coffee extract include coffee bean, coffee, coffee berry, coffee fruits. When coffeic acid is present non-limiting sources of coffeic acid suitable for use in the present invention include tea, berries, coffee bean, coffee, coffee berry, coffee fruits, rosemary extract, and/or grape extract.

When coffeic acid is present, the compositions can comprise from about 0.01% to about 90%, alternatively from about 0.1% to about 35%, alternatively from about 1% to about 15%, alternatively from about 1% to about 10%, and alternatively from about 3% to about 10% coffeic acid, by weight of the composition.

### Turmeric Extract

The compositions of the present invention can comprise turmeric extract. Turmeric extract contains polyphenols. Turmeric extract is a spice which comprises a main active compound that is curcumin. Curcumin is a bioactive polyphenol plant pigment. Without being limited by theory, it is believed that curcumin has antioxidant activity and can be beneficial in enhancing immune response to a respiratory condition. A non-limiting source of turmeric extract for use in the present invention is tumeric.

When present, the compositions can comprise from about 0.01% to about 90%, alternatively from about 0.1% to about 35%, alternatively from about 1% to about 15%, alternatively from about 1% to about 10%, and alternatively from about 3% to about 10% turmeric extract, by weight of the composition.

### Blueberry Extract

The compositions of the present invention can comprise blueberry extract. Blueberry extract contains polyphenols. Blueberry extract is rich in anthocyanins which display antioxidant activity by quenching singlet oxygen, and can be beneficial in enhancing immune response to a respiratory condition. A non-limiting source of blueberry extract for use in the present invention is blueberry.

When present, the composition can comprise from about 0.01% to about 90%, alternatively from about 0.1% to about 35%, alternatively from about 1% to about 15%, alternatively from about 1% to about 10%, and alternatively from about 3% to about 10% blueberry extract, by weight of the composition.

### Grapeseed Extract

The compositions of the present invention can comprise grapeseed extract. Grapeseed extract contains polyphenols. Grape seed extract is rich in procyanidins which display antioxidant activity, which can be beneficial in enhancing immune response to a respiratory condition. Grape seed extract comprises about 38.5% procyanidins. A non-limiting source of grapeseed extract for use in the present invention is grape seed.

When present, the compositions can comprise from about 0.01% to about 90%, alternatively from about 0.1% to about 35%, alternatively from about 1% to about 15%, alternatively from about 1% to about 10%, and alternatively from about 3% to about 10% grapeseed extract, by weight of the composition.

### Other Plant-Derived Components

The compositions of the present invention can also comprise other plant-derived components that are known to have beneficial effects with respect to enhancing immune response to respiratory conditions. Non-limiting examples of other plant-derived components include: Andrographis (Andrographis paniculata), borage seed oil (Borago officinalis), sage (Salvia officinalis, Salvia lavandulaefolia, Salvia lavandulifolia), Astragalus (Astragalus membraneceus), Boneset (Eupatorium perfoliatum), Chamomile (Matricaria recutita, Chamaemelum nobile), Cordyceps (Cordyceps sinensis), Echinacea (Echinacea angustifolia DC, Echinacea pallida, Echinacea purpurea), Elder (Sambucas nigra L.), Euphorbia, Garlic (Allium sativum L.), Ginsing (American ginsing, Asian ginsing, Chinese ginsing, Korean red ginsing, Panax ginsing: Panax ssp. Including P. ginsing C.C. Meyer, and P. quinquefolius L.), Goldenseal (Hydrastis canadensis L.), Greater celandine (Chelidonium majus), Horseradish (Armoracia rusticana, Cochlearia armoracia), Kiwi (Actinidia deliciosa, Actinidia chinensis), Mistletoe (Visvum album L.), Peppermint/Peppermint oil (Mentha×peperita L.), Propolis, Slippery elm (Ulmus rubra Muhl, Ulmus fulva Michx), Sorrel (Rumex acetosa L., Rumex acetosella L.), Thyme/Thymus extract (Thymus vulgaris L.), Wild indigo (Baptisia australis), and combinations and/or mixtures thereof.

Some non-limiting examples of plant-derived components particularly useful with the present invention are described below.

### Andrographis Paniculata

The compositions of the present invention can comprise an andrographis extract, an active component thereof, or mixtures thereof. As used herein, the andrographis is a plant of the genus Andrographis, having a limited number of species within this genus largely present in Asia. Only a few of the species are medicinal. In one embodiment, the plant is of the species Andrographis paniculata, which may be referenced as Kalmegh in Ayurvedic medicine. Andrographis is typically standardized by quantifying the total amount of andrographolides, which often make up 5 to 20% of the extract.

Andrographis aids in reducing to an extent the symptoms or duration of colds. Without being limited by theory, it is believed that andrographis paniculata reduces the levels of inflammatory cytokines and chemokines, such as IP-10. Andrographolide, the principal component of andrographis, is remarkably similar in chemical structure to Vitamin D. Therefore, andrographis may provide some of its benefits by acting as a ligand at the Vitamin D receptor.

When present, the compositions can comprise Andrographis paniculata in amounts from about 5 mg to about 50 mg, alternatively from about 10 mg to about 40 mg, and alternatively from about 15 mg to about 30 mg of andrographolides, per daily dose of the composition.

### Allium Sativum

The compositions of the present invention can comprise Allium Sativum (garlic). Allium Sativum has been shown to be effective at reducing many of the cytokines and chemokines involved in the immune response to viral infections. Therefore, Allium Sativum can improve the symptoms of colds and flu by reducing the inflammatory cytokines and chemokines that have been shown to play a major role in producing symptoms. A combination of Allium Sativum, and/or Allicin, a component of Allium sativum, in the compositions of the present invention should provide extensive relief of colds and flu symptoms.

When present, the compositions can comprise from about 0.01% to about 90%, alternatively from about 0.1% to about 35%, alternatively from about 1% to about 15%, alternatively from about 1% to about 10%, and alternatively from about 3% to about 10% of Allium sativum, by weight of the composition.

When present, the compositions can comprise from about 100 mg to about 10,000 mg, alternatively from about 200 mg to about 5000 mg, and alternatively from about 500 mg to about 2000 mg of Allium sativum, per daily dose of the composition.

When present, the compositions can comprise from about 1000 μg to about 100,000 μg, alternatively from about 2000 μg to about 50,000 μg, and alternatively from about 5000 μg to about 20,000 μg of Allicin, per daily dose of the composition.

### Fungal-Derived Component HERE

Various preparations of fungi (such as mushrooms) and/or components thereof have traditionally been marketed and used for years in countries such as Japan. Such compositions have been shown to have various health benefits from enhancing well-being to providing immunomodulatory effects, and have been widely used for medicinal and health purposes. However, traditional preparations, such as for example a ground preparation, of such compositions may not allow the components thereof to be fully absorbed or utilized. Additionally, the components contained in such preparations are often generally not fully understood or identified. More recently, investigation into the active components of such traditional compositions has become an area of interest, particularly with respect to their effects on the immune system.

Therefore, the compositions of the present invention can also comprise fungal-derived components that are known to have beneficial effects with respect to enhancing immune response to respiratory conditions. A fungal-derived component can be a particularly preferred additional component of the invention. Non-limiting examples of such fungal-derived components include: Maitake and Shiitake mushrooms (Grifola frondosa and Lentinus edodes respectively) and Reishi mushroom (Ganoderma lucidum); yeast (Saccharomyces cerevisiae, Saccharomyces boulardii) and cell wall extracts of yeast cells; and molds (Aspergillus). The fungal-derived components can include whole, ground, crude-sized, and superfine particle extracts, micellary extracts, and mixtures thereof, of the fungus.

A particularly useful fungal-derived component can comprise an extract of edible mushroom. Useful mushrooms are those fungi capable of forming fruit body and include but are not limited to: Lentinus edodes, Pleurotus ostreatus, Pholiota nameko, Flammulina velutipes, Tricholoma matsutake, Lyophyllum shimeji, Schizophyllum commune, Crepitodus variabilis, Lyophyllum ulmarinum, Grifola umbellate, Grifola frondosa, Coriolus versicolor, Fomes fomentarius, Volvavella volvacea, Auricularia aurcula-judae, Ganoderma lucidum, Ganoderma appanatum, Fomitopsis pinicola, Dictyophora indusiata, Sparassis crispa, Agaricus blazei, Peziza vesiculosa, and mixtures thereof. Lentinus edodes is a particularly useful non-limiting example thereof. The compositions can comprise an extract, active components thereof, purified components thereof, and mixtures/combinations thereof.

Of particular use in the present invention are extracts of shiitake mushroom. A micellar preparation of superfine particle extract of shiitake mushroom has been shown to be effective at providing an anti-allergy effect in mice. For example, US 2004/0142000 describes a hot water extract of mushroom which was pulverized to prepare superfine particles which were then prepared as micells and dispersed, for example in water. Such superfine particles had an average particle size diameter of about 10 μm or less, most preferably from 0.01 to 1 μm, in a micellar state, and showed improved incorporation and absorption through mucosa versus non-micellar preparations. Methods of preparing micellar extracts of mushroom are described in US 2004/014200.

An embodiment of the present invention can comprise superfine particle extracts of shiitake mushroom. An embodiment of the present invention can also comprise a micellar preparation of superfine particles of shiitake mushroom extract. It is believed, without limitation, that a micellar preparation of superfine particles can prevent the superfine particles from re-aggregating, and thus provide for improved availability and absorption, and therefore effectiveness, of the particles versus non-micellar preparations.

Thus, embodiments of the present invention can comprise superfine particle extracts of fungal-derived components and/or micellar preparations of superfine particle extracts fungal-derived components.

When such superfine particle extract of shiitake mushroom extract is present, the compositions can comprise from about 0.01 mg to about 50 g, alternatively from about 1 mg to about 50 g, alternatively from about 10 mg to about 10 g, alternatively from about 10 mg to about 1 g, and alternatively about 15 mg of superfine extract, per day, of the composition.

Another embodiment of the invention can comprise superfine particles of beta-glucan, prepared as, for example, micellar beta-glucan, which can be derived from shiitake mushroom. Beta-1,3-glucan has been known to improve the life of non-operable, recurrent gastric cancer patents, and is marketed for such use intravenously. However, beta-1,3-glucan was ineffective orally for such use. Beta-1,3-glucans generally have a particle size of about 100-200 μm in aqueous solution, which hampered absorption through the abdominal mucosa. Additionally, the particle size impaired the immunostimulating effect of the beta-1,3-glucan. However, it has been found that preparations of superfine particles of beta-1,3-glucan with a particle size of about 0.2 μm are able to pass through the mucosal barrier.

For example a purified beta-1,3-glucan extract having superfine particles of beta-1,3-glucan, has been shown to be effective at reducing symptoms of Japanese cedar pollen allergy. Alleviation of Seasonal Allergic Symptoms with Superfine beta-1,3-glucan: A Randomized Study. Yamada, J., Hamuro J., Hatanaka, H., Hamabata K., Kinoshita S., J. Allergy Clin Immunol. 2007 May; 119 (5): 1119-26. Epub 2007 Mar. 26. Additionally, preparation and use of micellar beta-glucan extract of shiitake mushroom is described in US 2004/0142000. Beta-glucans, including beta-1,3-glucan and beta-1,6-glucan, can also be obtained from sources other than shiitake mushroom, and can be prepared as superfine particles and or micellar preparations. An example of a purified beta-1,3-glucan is known as Mitherapist®, and is available from Ajinomoto Co. Inc., Japan.

Thus, the present invention can comprise superfine particle extracts of beta-glucans and/or micellar preparations of superfine particle extracts of beta-glucans.

When superfine beta-glucan is present, the compositions can comprise from about 0.01 mg to about 50 g, alternatively from about 1 mg to about 50 g, alternatively from about 10 mg to about 10 g, alternatively from about 10 mg to about 1 g, and alternatively about 15 mg of superfine beta-glucan, per day of the composition.

Superfine particles, as used herein, have an average particle diameter of about 10 μm or less, alternatively 1 μm or less, alternatively from about 0.01 μm to 1 μm, as determined after being dispersed in water. Average particle diameter can be readily determined with a particle size distribution meter.

### Amino Acids

The compositions of the present invention can comprise an amino acid. Amino acids are the “building blocks” of the body. Besides building cells and repairing tissue, they form antibodies to combat invading bacteria & viruses; they are part of the enzyme & hormonal system; they build nucleoproteins (RNA & DNA); they carry oxygen throughout the body and participate in muscle activity. When protein is broken down by digestion the result is 22 known amino acids. Eight are essential (cannot be manufactured by the body) the rest are non-essential (i.e. can be manufactured by the body with proper nutrition).

When an amino acid is present, the amino acid is selected from the group consisting of 1-Tryptophan, Taurine, Histidine, Carnosine, Alanine, Cysteine, and mixtures and/or combinations thereof.

### 1-Tryptophan

The compositions of the present invention can comprise 1-tryptophan. Not to be limited by theory, the inflammatory cytokines and chemokines produced during a respiratory condition may be responsible for the malaise and fatigue associated with respiratory conditions through their interaction with the olfactory bulb of the nasal passages, and ultimately with the Hypothalamus-Pituitary-Adrenal (HPA) axis. The HPA axis is the seat of many physiological functions, playing a major role in controlling mood. Serotonin is a mediator that acts on the HPA axis and is correlated with mood. Tryptophan, the metabolic precursor to serotonin, is degraded by cytokines that are unleashed during a respiratory condition such as an upper respiratory tract viral infection. Therefore tryptophan levels may be lowered during a cold, leading to worsened mood, malaise and a feeling of fatigue. A composition comprising 1-tryptophan can provide a mechanism by which the HPA axis could improve the malaise and fatigue associated with respiratory conditions, and hence provide a meaningful benefit in the relief of symptoms of respiratory conditions.

When present, the compositions can comprise from about 250 mg to about 2500 mg, alternatively from about 300 mg to about 2000 mg, and alternatively from about 400 mg to about 1000 mg of 1-tryptophan, per daily dose of the composition.

### Taurine

The compositions of the present invention can comprise taurine. Taurine is an amino acid used as a building block of all the other amino acids; it is found in the eye, heart muscle, white blood cells, skeletal muscle, and central nervous system. Taurine and sulfur are considered to be factors that aid in the clearing of free radical wastes, and it has been suggested that taurine can provide relief from fatigue which is a common symptom of respiratory conditions. Thus, without being limited by theory, it is believed that taurine can enhance immune response to a respiratory condition.

When present, the compositions can comprise at least about 0.05%, alternatively from about 0.05% to about 10%, and alternatively from about 0.1% to about 5% taurine, by weight of the composition.

### Histidine

The compositions of the present invention can comprise histidine. Histidine is found abundantly in hemoglobin. Histidine is needed for growth and for the repair of tissue, as well as the maintenance of the myelin sheaths that act as protector for nerve cells. Histidine is further required for the manufacture of both red and white blood cells, and helps to protect the body from damage caused by radiation and in removing heavy metals from the body. Histidine is a precursor of histamine, a compound released by immune system cells during an allergic reaction, and thus can aid in enhancing immune response to a respiratory condition.

When present, the compositions can comprise at least about 0.05%, alternatively from about 0.05% to about 10%, and alternatively from about 0.1% to about 5% histidine, by weight of the composition.

### Carnosine

The compositions of the present invention can comprise carnosine. Carnosine has excellent potential to act as a natural antioxidant with hydroxyl radical, singlet oxygen scavenging and lipid peroxidase activities. It is believed that Carnosine may reduce the destruction of valuable proteins and DNA by sugar molecules, a process known as glycosylation. Carnosine may help prevent damage from glycosylation, ridding the system of any abnormal substances and leaving it free to function optimally, thus, without being limited by theory, can aid in enhancing immune response to a respiratory condition.

When present, the compositions can comprise at least about 0.05%, alternatively from about 0.05% to about 10%, and alternatively from about 0.1% to about 5% carnosine, by weight of the composition.

### Alanine

The compositions of the present invention can comprise alanine. Alanine is a nonessential amino acid that can be manufactured by mammals from other sources as needed. Alanine is one of the simplest of the amino acids and is involved in the energy-producing breakdown of glucose. In conditions of sudden anaerobic energy need, when muscle proteins are broken down for energy, Alanine acts as a carrier molecule to take the nitrogen-containing amino group to the liver to be changed to the less toxic urea, thus preventing buildup of toxic products in the muscle cells when extra energy is needed. Alanine is found in a wide variety of foods, but is particularly concentrated in meats.

When present, the compositions can comprise at least about 0.05%, alternatively from about 0.05% to about 10%, and alternatively from about 1% to about 5% alanine, by weight of the composition.

### Cysteine

The compositions of the present invention can comprise cysteine. Cysteine functions as an antioxidant, and can deactivate free radicals and neutralize toxins. Thus, without being limited by theory, cysteine can aid in enhancing immune response to a respiratory condition.

When present, the compositions comprise at least about 0.05%, alternatively from about 0.05% to about 10%, and alternatively from about 0.2% to about 5% cysteine, by weight of the composition.

### Carotenoids

The composition of the present invention can comprise a carotenoid. A “carotenoid” is a class of pigments occurring in the tissues of higher plants, algae, bacteria and fungi. They are usually yellow to deep red crystalline solids, soluble in fats and oils, insoluble in water, high-melting, stable to alkali, unstable to acids and oxidizing agents, their color is easily destroyed by hydrogenation or by oxidation, and some are optically active. Carotenoids are natural pigments synthesized by plants and microorganisms that are thought to function as light absorbing pigments during photosynthesis and to protect cells from photosensitization. Structurally, carotenoids consist of eight isoprenoid units joined so that their arrangement is reversed at the center of the molecule. Carotenoid structure strongly affects the physical properties, chemical reactivity and biologic functions of these compounds. It has been suggested that the size, shape, hydrophobicity and polarity of individual carotenoids may dramatically affect the bioavailability, absorption, circulation, tissue and subcellular distribution and excretion in mammals. Carotenoids have demonstrated antioxidant activity and as well as other biological activities in addition to maintaining oxidative balance, thus without being limited by theory, it is believed that carotenoids can aid in enhancing immune response to a respiratory condition.

When a carotenoid is present, the carotenoid is selected from the group consisting of lutein, astaxanthin, zeaxanthin, bixin, lycopene, and mixtures thereof.

### Lutein

The compositions of the present invention can comprise lutein. Lutein is a powerful antioxidant. Lutein and zeaxanthin are structural isomers of one another. Lutein can be extracted in crystalline form from marigolds. Dietary sources of lutein include mustard greens, spinach, kale, broccoli, leaf lettuce, green peas, brussel sprouts, corn, some squash and green beans.

When present, the compositions can comprise at least about 0.01%, alternatively from about 0.01% to about 20%, and alternatively from about 0.05% to about 10% lutein, by weight of the composition.

### Zeaxanthin

The compositions of the present invention can comprise zeaxanthin. Zeaxanthin is also a powerful antioxidant. Zeaxanthin can be extracted in crystalline form from marigolds. Dietary sources of zeaxanthin include mustard greens, spinach, kale, broccoli, leaf lettuce, green peas, brussel sprouts, corn, some squash and green beans.

When present, the compositions can comprise at least about 0.01%, alternatively from about 0.01% to 20%, and alternatively from about 0.05% to about 10% zeaxanthin, by weight of the composition.

### Astaxanthin

The compositions of the present invention can comprise astaxanthin. Astaxanthin is also a powerful antioxidant and can be provided as free astaxanthin or as astaxanthin diester. Naturally produced astaxanthin can be obtained from fungi, crustaceans, and algae, e.g., Haematococcus sp. (e.g., as described in U.S. Pat. No. 5,744,502). Astaxanthin is also produced by wild-type and genetically engineered Pfaffia yeast, and is commercially available from Archer Daniels Midland Co.; Aquasearch Inc.; AstaCarotene AB; Cyanotech Corporation and Micro Gaia, Inc. Synthetically produced astaxanthin is also commercially available from Hoffman-LaRoche, Ltd.

When present, the compositions can comprise at least about 0.01%, alternatively from about 0.01% to about 20%, and alternatively from about 0.05% to about 10% astaxanthin, by weight of the composition.

### Bixin

The compositions of the present invention can comprise bixin. Bixin is a naturally occurring carotenoid, found in the pulp of the B. orellana seed (also called annatto seed), used all over the world as a red-orange dye for coloring rice, cheeses, soft drinks, oil, butter, soup and cosmetics. As annatto extract, it is used as a color additive in food. Bixin is an antioxidant that can scavenge free radicals and prevent oxidative damage.

When present, the compositions can comprise at least about 0.01%, alternatively from about 0.01% to about 20%, and alternatively from about 0.05% to about 10% bixin, by weight of the composition.

### Lycopene

The compositions of the present invention can comprise lycopene. Lycopene is an open-chain unsaturated carotenoid that imparts red color to tomatoes, guava, rosehip, watermelon and pink grapefruit. Lycopene is a proven antioxidant, which neutralize free radicals that may damage the body's cells.

When present, the compositions can comprise at least about 0.01%, alternatively from about 0.01% to about 20%, and alternatively from about 0.05% to about 10%, lycopene, by weight of the composition.

### Antioxidants

The composition of the present invention can comprise an antioxidant in addition to the vitamins, plant-derived components, elements, and carotenoids described above as having antioxidant properties. An antioxidant is an enzyme or other organic molecule that can counteract the damaging effects of oxygen in tissues. Although the term technically applies to molecules reacting with oxygen, it is often applied to molecules that protect from any free radical. Antioxidants can include natural and synthetic vitamins, plant-derived components and carotenoids, many of which have been described above, in addition to the antioxidants described in this section.

When an antioxidant is present, non-limiting examples of such antioxidants include tocopherols (Vitamin E), Vitamin C (described above), Vitamin A (described above), CoQ10, plant-derived materials (described above), carotenoids (described above), selenium (described above), and mixtures thereof.

### Vitamin E

The compositions of the present invention can comprise Vitamin E. Vitamin E is a lipid soluble compound and the most significant antioxidant activity of vitamin E is localized to cellular membranes. Vitamin E maintains oxidative balance by protecting cellular membranes from lipid peroxidation. Vitamin E is a lipid soluble antioxidant and provides defenses against cellular oxidative damage. Major dietary sources of vitamin E are vegetable oils, margarine and shortening, with nuts, seeds, whole grains and wheat germ providing additional sources. The term “Vitamin E” typically includes eight different chemical forms: four tocopherols and four tocotrienols. The most biologically active form of vitamin E is alpha-tocopherol.

When present, the compositions can comprise from about 1 mg to about 1000 mg, alternatively from about 1 mg to about 800 mg, and alternatively from about 2 mg to about 200 mg of vitamin E, per daily dose of the composition.

When present, the compositions can comprise at least about 0.01%, alternatively from about 0.01% to about 10%, and alternatively from about 0.2% to about 5% Vitamin E, by weight of the composition.

### Coenzyme Q10

The compositions of the present invention can comprise coenzyme Q10 (CoQ10). Coenzyme Q10 is a powerful naturally occurring compound that promotes chemical reactions and aids in protecting mammals from free radicals. It is also called ubiquinone. Coenzyme Q10 (CoQ10) is naturally present in foods, and can be synthesized by mammals from the amino acid tyrosine during a multistage (17 stages) process requiring eight vitamins and several trace elements. Coenzyme Q10 provides antioxidant qualities as well as the control it exercises on the flow of oxygen within cells, assistance with cardiovascular functioning, the production of energy, assistance with absorption of other nutrients as well as having immune boosting properties.

When present, the compositions can comprise at least about 0.01%, alternatively from about 0.01% to about 10%, and alternatively from about 0.2% to about 5% Coenzyme Q10, by weight of the composition.

When present, the compositions can comprise from about 1 mg to about 400 mg, alternatively from about 2 mg to about 400 mg, and alternatively from about 3 mg to about 300 mg of Coenzyme Q10, per daily dose of the composition.

### Respiratory Actives

The compositions of the present invention can also comprise one or more of a wide range of common respiratory actives. Non-limiting examples include decongestants, anticholinergics, analgesics, anti-inflammatories, antipyretics, antivirals, antitussives, expectorants, mucolytics, and antihistamines and non-sedating antihistamines, and mixtures and/or combinations thereof. Other additional respiratory actives can include local anesthetics, demulcents, herbal remedies, supplements, natural ingredients, energy boosting ingredients, sleep aids, and mixtures and/or combinations thereof.

Non-limiting examples of decongestants include oxymetazoline, phenylephrine, xylometazoline, naphazoline, 1-desoxyephedrine, ephedrine, propylhexedrine, pseudoephedrine, and phenylpropanolamine.

Non-limiting examples of anticholinergics include ipratropium, chlorpheniramine, brompheniramine, diphenhydramine, doxylamine, clemastine, and triprolidine.

Non-limiting examples of analgesics, anti-inflammatories and antipyretics include ibuprofen, ketoprofen, diclofenac, naproxen, acetaminophen, and aspirin.

Non-limiting examples of antivirals include: amantidine, rimantidine, pleconaril, zanamivir, and oseltamivir.

Non-limiting examples of antitussives include codeine, dextromethorphan, chlophedianol and levodropropizine.

A non-limiting example of expectorants includes guaifenesin.

Non-limiting examples of mucolytics include ambroxol and N-acetylcysteine.

Examples of antihistamines include diphenhydramine, doxylamine, triprolidine, clemastine, pheniramine, chlorpheniramine, brompheniramine, Dexbrompheniramine, loratadine, cetirizine and fexofenadine, Amlexanox, Alkylamine Derivatives, Cromolyn, Acrivastine, Ibudilast, Bamipine, Ketotifen, Nedocromil, Omalizumab, Dimethindene, Oxatomide, Pemirolast, Pyrrobutamine, Pentigetide, Thenaldine, Picumast, Tolpropamine, Ramatroban, Triprolidine, Repirinast, Suplatast Tosylate Aminoalkylethers, Tazanolast, Bromodiphenhydramine, Tranilast, Carbinoxamine, Traxanox, Chlorphenoxamine, Diphenhydramine, Diphenylpyaline, Doxylamine, Embramine, p-Methyldiphenhydramine, Moxastine, Orphenadrine, Phenyltoloxamine, Setastine, Ethylenediamine Derivatives, Chloropyramine, Chlorothen, Methapyrilene, Pyrilamine, Talastine, Thenyldiamine, Thonzylamine Hydrochloride, Tripelennamine, piperazines, Chlorcyclizine, Clocinizine, Homochlorcyclizine, Hydroxyzine, Tricyclics, Phenothiazines, Mequitazine, Promethazine, Thiazinamium Methylsulfate, Other Tricyclics, Azatadine, Cyproheptadine, Deptropine, Desloratadine, Isothipendyl, Olopatadine, Rupatadine, Antazoline, Astemizole, Azelastine, Bepotastine, Clemizole, Ebastine, Emedastine, Epinastine, Levocabastine, Mebhydroline, Mizolastine, Phenindamine, Terfenadine, and Tritoqualine.

The when present, the compositions can comprise from about 0% to about 20%, alternatively from about 0.0001% to about 15%, alternatively from about 0.001% to about 10%, and alternatively from about 0.01% to about 5% of the respiratory active, by weight of the composition.

When present, the compositions can comprise from about 0.001 mg to about 1000 mg, alternatively from about 2.5 mg to about 750 mg, and alternatively from about 5 mg to about 600 mg of the respiratory active, per dose of the composition. Total daily dosage amounts of respiratory actives depend on the particular respiratory active, as would be understood by one of skill in the art. Example recommended daily dosage amounts of common respiratory actives can be found in US FDA guidance entitled OTC Cough/Cold Drug Products: 21 CFR Part 341 accessible at http://www.fda.gov/eder/drug/unapproved drugs/cough cold products.pdf, or Beradi, Rosemary, et al. (Editors) Handbook of Nonprescription Drugs, 15^{th} Edition (2006) American Pharmacists Association, Washington, D.C., USA.

### Optional Ingredients

The compositions of the present invention can also comprise one or more of a wide range of optional ingredients and process aids. Non-limiting examples of optional ingredients include natural ingredients, plasticizers, colorants, flavorants, sweeteners, buffering agents, slip aids, excipients, carriers, and mixtures and/or combinations thereof. Other optional ingredients can include, stabilizers, biological additives such as enzymes (including proteases and lipases), chemical additives, coolants, chelants, denaturants, drug astringents, emulsifiers, external analgesics, fragrance compounds, humectants, opacifying agents (such as zinc oxide and titanium dioxide), anti-foaming agents (such as silicone), preservatives (such as butylated hydroxytoluene (BHT) and butylated hydroxyanisole (BHA), propyl gallate, benzalkonium chloride, EDTA, benzyl alcohol, potassium sorbate, parabens and mixtures thereof), reducing agents, solvents, hydrotropes, solublizing agents, suspending agents (non-surfactant), solvents, viscosity increasing agents (aqueous and non-aqueous), sequestrants, keratolytics, and the like, and mixtures and/or combinations thereof.

Generally, unless otherwise specified herein, when present, the compositions comprise from about 0.001% to about 99%, alternatively from about 0.01% to about 80%, alternatively from about 0.01% to about 50%, and alternatively from about 0.01% to about 10%, of optional ingredient(s) by weight of the composition.

### Natural Ingredients

The composition of the present invention can comprise one or more of a wide range of natural ingredients. Non-limiting examples of such natural ingredients include animal protein, plant protein, farinaceous matter, vegetables, fruit, egg-based materials, undenatured proteins, food grade polymeric adhesives, gels, polyols, starches, gums, seasonings, salts, time-release compounds, aroma modifiers, textured wheat protein, textured soy protein, textured lupin protein, textured vegetable protein, fatty acids, and combinations thereof. Particularly useful natural ingredients are exemplified below.

### Fruit

The compositions of the present invention can comprise, as an optional natural ingredient, at least one fruit, fruit flavor, and/or fruit juice and/or extract. Non-limiting examples include tomatoes, apples, avocado, pears, peaches, cherries, apricots, plums, grapes, oranges, grapefruit, lemons, limes, cranberries, raspberries, blueberries, watermelon, cantaloupe, mushmelon, honeydew melon, strawberries, banana, and combinations thereof.

### Fatty Acids

The compositions of the present invention can comprise a fatty acid. Long chain fatty acids play a key role in arachidonic acid metabolism which could be useful in the modulation of pain and inflammation. Currently, long chain fatty acids, such as omega-6 fatty acids are used for their antioxidant and immune health benefits.

Non-limiting examples of suitable long chain fatty acids include alpha-linoleic acid, gamma linolenic acid, linoleic acid, eicosapentanoic acid, and docosahexanoic acid. Fish oils are a suitable source of eicosapentanoic acids (EPA) and docosahexanoic acid (DHA).

When present, the compositions can comprise from at least about 0.05%, alternatively at least about 0.1%, and alternatively at least about 0.15% DHA, by weight of the composition.

When present, the compositions can comprise from at least about 0.05%, alternatively at least about 0.1%, and alternatively at least about 0.15% EPA, by weight of the composition.

### Plasticizers

The compositions of the present invention can comprise a plasticizer. Without intending to be limited by theory, plasticizers cause a composition to become more easily deformed, less brittle, or less prone to mechanical damage. Thus, one or more plasticizers may optionally be added to the present compositions as desired.

Non-limiting examples of plasticizers include phthalates (e.g., diethyl phthalate, dibutyl phthalate, dioctyl phthalate), citrates (e.g., triethyl citrate (e.g., CITROFLEX 2), acetyl triethyl citrate, tributyl citrate, and acetyl tributyl citrate), polyhydric alcohols, (e.g., sorbitol, glycerol), triacetin (glyceryl triacetate), polyethylene glycol (e.g., CARBOWAX 400), polysorbate 80, acetylated monoglycerides, glycerol, propylene glycol, fatty acid esters, surfactant polymers, camphor, silicone oil, castor oil, and mixtures thereof.

The amount of plasticizer used will vary, for example depending on the plasticizer used and the desired character of the composition. When present, the compositions can comprise from about 0.001% to about 20%, alternatively from about 0.01% to about 15%, and alternatively from about 0.1% to about 10% of a plasticizer, by weight of the composition.

### Colorants

The compositions of the present invention can comprise a colorant. One or more pigments or other suitable colorants, such as dyes and lakes, can be incorporated into the compositions. For example, U.S. FD&C dyes (e.g., yellow #5, blue #2, red #40) or U.S. FD&C lakes can be used. Illustrative lakes which can be used in the present invention include, for example, Lake red #40, yellow #6, blue #1, and the like. Additionally, a mixture of U.S. FD&C dyes and/or U.S. FD&C lakes in combination with other conventional food and food colorants may be used. As further examples, riboflavin and beta-carotene can also be used as colorants. Additionally, other natural agents can be utilized as colorants, including, for example, fruit, vegetable, or plant extracts such as grape, black currant, aronia, carrot, beetroot, red cabbage, and hibiscus, and mixtures and/or combinations thereof.

The amount of colorant used will vary, depending on the agents used and the character or intensity desired in the finished composition. One of ordinary skill in the art can readily make such determination.

When present, the compositions can comprise from about 0.0001% to about 5%, alternatively from about 0.001% to about 1%, and alternatively from about 0.005% to about 0.1% of a colorant, by weight of the composition.

### Flavorants

The compositions of the present invention can comprise a flavorant. One or more flavorants can be incorporated in the compositions herein in order to enhance their palatability. Flavorants can be particularly important in compositions to be administered to children. Any natural or synthetic flavorant and/or mixtures and/or combinations thereof can be used in the present invention. Particularly suitable for use in the present invention are fruit flavors. These fruit flavors can be derived from natural sources such as fruit juices and flavor oils, or may alternatively be synthetically prepared.

Non-limiting examples of suitable flavors are exotic and lactonic flavors including, for example, passion fruit flavors, mango flavors, pineapple flavors, cupuacu flavors, guava flavors, cocoa flavors, papaya flavors, peach flavors, and apricot flavors. In addition, a variety of other fruit flavors can be utilized, non-limiting examples of which include, apple flavors, citrus flavors, grape flavors, raspberry flavors, cranberry flavors, cherry flavors, grapefruit flavors, and the like.

Non-limiting examples of additional flavorants and mixtures and/or combinations thereof include vanilla, honey lemon, lemon honey, cherry vanilla, peach, honey ginger, cherry, cherry cream, mint, vanilla mint, dark berry, black berry, raspberry, peppermint, spearmint, honey peach, acai berry, cranberry, honey cranberry, tropical fruit, dragon fruit, wolf berry, red stem mint, pomegranate, black current, strawberry, lemon, lime, peach ginger, orange, orange cream, cream sickle, apricot, anethole, ginger, jack fruit, star fruit, blueberry, fruit punch, lemon grass, chamomile lemon grass, lavender, banana, strawberry banana, grape, blue raspberry, lemon lime, coffee, espresso, cappuccino, honey, wintergreen mint, bubble gum, tart honey lemon, sour lemon, green apple, boysenberry, rhubarb, strawberry rhubarb, persimmon, green tea, black tea, red tea, white tea, honey lime, cherry lime, apple, tangerine, grapefruit, kiwi, pear, vanillin, ethyl vanillin, maltol, ethyl-maltol, pumpkin, carrot cake, white chocolate raspberry, chocolate, white chocolate, milk chocolate, dark chocolate, chocolate marshmallow, apple pie, cinnamon, hazelnut, almond, cream, crème brule, caramel, caramel nut, butter, butter toffee, caramel toffee, aloe vera, whiskey, rum, cocoa, licorice, pineapple, guava, melon, watermelon, elder berry, mouth cooler, raspberries and cream, peach mango, tropical, cool berry, lemon ice, nectar, spicy nectar, tropical mango, apple butter, peanut butter, tangerine, tangerine lime, marshmallow, cotton candy, apple cider, orange chocolate, and mixtures and/or combinations thereof.

The amount of flavorant used will vary, depending on the flavorants used and the character or intensity of flavor desired in the finished composition. One of ordinary skill in the art can readily make such determination.

### Sweeteners

The compositions of the present invention can comprise one or more sweeteners, including for example carbohydrate sweeteners and natural or artificial no/low calorie sweeteners. For example, the compositions used herein can be sweetened with any of the common carbohydrate sweeteners, such as monosaccharides or disaccharides. Non-limiting examples of sugar sweeteners suitable for use in the compositions of the present invention include sucrose, fructose, glucose, maltose, and mixtures and/or combinations thereof.

One or more high intensity and/or artificial sweeteners can also be utilized. Non-limiting examples of such sweeteners include saccharin and its salts, cyclamates, L-aspartyl-L-phenylalanine lower alkyl ester sweeteners (e.g. aspartame); L-aspartyl-D-alanine amides; L-aspartyl-D-serine amides; L-aspartyl-L-1-hydroxymethylalkaneamide sweeteners; L-aspartyl-1-hydroxyethyalkaneamide sweeteners; L-aspartyl-D-phenylglycine ester and amide sweeteners; N—[N-3,3-dimethylbutyl)-L-alpha-aspartyl]-L-phenylalanine 1-methyl ester sweeteners; thaumatin; dihydrochalcones; cyclamates; steviosides; glycyrrhizins, synthetic alkoxy aromatics; sucralose; suosan; miraculin; monellin; sorbitol, xylitols; talin; cyclohexylsulfamates; substituted imidazolines; synthetic sulfamic acids such as acesulfame, acesulfame K and n-substituted sulfamic acids; oximes such as perilartine; peptides such as aspartyl malonates and succanilic acids; dipeptides; amino acid based sweeteners such as gem-diaminoalkanes, meta-aminobenzoic acid, L-aminodicarboxylic acid alkanes, and amides of certain alpha-aminodicarboxylic acids and gem-diamines; and 3-hydroxy-4-alkyloxyphenyl aliphatic carboxylates or heterocyclic aromatic carboxylates; erythritol; and mixtures and/or combinations thereof.

The amount of sweetener used can vary, depending on the sweetener used and the character or intensity of sweetness desired in the finished composition. One of ordinary skill in the art can readily make such determinations.

When a sweetener is present, the compositions can comprise from about 0.0001% to about 90%, alternatively from about 0.0001% to about 70%, alternatively from about 0.0001% to about 50%, alternatively from about 0.0001% to about 20%, alternatively from about 0.0001% to about 10%, and alternatively from about 0.0001% to about 5%, of the sweetener, by weight of the composition.

When an artificial sweetener is present, the compositions can comprise from about 0.0001% to about 5%, alternatively from about 0.0001% to about 3.5%, alternatively from about 0.0001% to about 2%, alternatively from about 0.0001% to about 1%, and alternatively from about 0.05% to about 1% artificial sweetener, by weight of the composition.

### Buffering Agents

One or more buffering agents can be utilized in the compositions of the present invention in order to, for example, maintain a constant pH within an environment. Such buffers can include acetate buffers, citrate buffers, and phosphate buffers. Non-limiting examples include acetic acid, sodium acetate, citric acid, sodium citrate, monobasic sodium phosphate, dibasic sodium phosphate, and sodium chloride.

The amount of buffer used can vary, depending on the buffering agents used and the effect desired in the finished composition. One of ordinary skill in the art can readily make such determinations.

### Slip Aids

One or more slip aids can optionally be included in the present compositions to improve surface friction, water resistance, abrasion resistance, or other mechanical properties of the composition. For example, a slip aid may be included on the surface of the composition, such that a mammal can more easily swallow the composition when orally administered.

Non-limiting examples of suitable slip aids include wax additives including, for example, animal, fossil, vegetable, mineral, or synthetic waxes. Preferred wax additives include carnuba, beeswax, carob, candelilla, ozocerite, polyethylene waxes, paraffin waxes, polypropylene waxes, and the like, and mixtures and/or combinations thereof. Other non-limiting examples include surfactants, glycerin, oils, and polyethylene glycols.

The amount of slip aid used can vary, depending on the slip aid used and the specific purpose of the slip aid. One of ordinary skill in the art cab readily make such determination.

### Excipients

The compositions of the present invention can comprise one or more excipients, non-limiting examples of which include disintegrants, fillers, diluents, lubricants, binding agents, coatings, sustained-release agents and compression aids. Non-limiting examples of particular excipients include microcrystalline cellulose (a filler/compression aid), dicalcium phosphate (a diluent/filler/compression aid), stearic acid (a lubricant), magnesium stearate (a lubricant), corn starch (a filler/compression aid), lactose (a filler), sodium croscarmellose (a disintegrant), sodium starch glycolate (a disintegrant), crospovidone (a disintegrant) polyvinylpyrollidone (a binding agent), methacrylic acid copolymer type C (an enteric polymer coating), hypromellose (a sustained-release matrix polymer), gelatin (a filler) and combinations thereof.

When present, the compositions can comprise from about 1% to about 99%, alternatively from about 2% to about 70%, alternatively from about 3% to about 40%, alternatively from about 5% to about 30%, and alternatively from about 6% to about 25% of the excipient, by weight of the composition.

### Carrier Systems

The compositions of the present invention can be administered orally as compositions comprising a pharmaceutically acceptable carrier system. The type of carrier system can depend on the desired dosage form. Any pharmaceutically acceptable carrier system in the form of a liquid, solid, or gas is suitable for the delivery of the compositions to enhance the immune system in response to a respiratory condition. Depending on the desired dose form of the composition and, where applicable, the delivery device to be used, the compositions of the present invention can optionally include a pharmaceutically acceptable carrier system, non-limiting examples of which include water, water-miscible solvents including ethanol, propylene glycol, polyethylene glycol, transcutol, glycerol, and other known or otherwise effective water-miscible solvents; liquid aerosol propellants; and mixtures and/or combinations thereof.

When the compositions of the present invention are administered in solid or liquid dosage forms, using a solid or liquid pharmaceutically acceptable carrier system, non-limiting examples of dose forms include the forms of powder, capsule or tablet, including enteric coated and sustained-release forms. When compositions of the present invention are to be dosed in the form of a powder they can be packaged in a sachet, or tubular form such as a stick pack or straw.

Pharmaceutically acceptable solid or liquid carrier systems for such dosage forms can be added to the compositions of the present invention to provide aid in processing of the compositions, to aid in the consistency of the compositions, to provide for improved stability, to facilitate handling, for hygroscopicity benefits, and so forth.

Pharmaceutically acceptable solid carrier system materials include ingredients such as particulate and powder fillers, for example, a lactose powder, a sucrose powder and/or mixtures thereof.

### Method of the Invention

The methods of the present invention comprise orally administering (i.e., through ingestion) a composition of the present invention to a mammal to treat a respiratory condition, preferably by enhancing immune response to a respiratory condition. The composition contains a probiotic strain of bacteria as described herein. In one embodiment, the mammal is a human, or may be a domestic animal such as a cat, dog, cow, rabbit, or horse. In one embodiment, the mammal is a human, and in particular, the mammal can be a human child.

The respiratory condition which is treated, and to which immune response is enhanced, by the invention is defined above and would be well understood by one of ordinary skill in the art. In one embodiment, the respiratory condition is selected from common cold, influenza, allergy, rhinitis, or sinusitis. As used herein and defined above, treatment of a respiratory condition includes the prevention, cure, mitigation, amelioration, inhibition, or alleviation of that condition, and/or the prevention, cure, mitigation or alleviation of any, some, or all of the symptoms associated with that condition. Symptoms may include, for example, sore throat, cough, fatigue, sneeze, running nose, stuffy nose, nasal drip, itchy nose, itchy eyes, watery eyes, excessive mucus, sinus pressure, and combinations thereof.

As used herein, the term “orally administering” with respect to the mammal means that the mammal ingests or a human is directed to administer, or does administer, to oneself (or another human or other animal) one or more of the compositions herein. Wherein the human is directed to administer the composition, such direction can be that which instructs and/or informs the human that use of the composition may and/or will provide the referenced benefit, for example, alleviation of one or more symptoms associated with the common cold or influenza. For example, such direction may be oral direction (e.g., through oral instruction from, for example, a physician, pharmacist, nurse, veterinarian or other health professional), radio or television media (i.e., advertisement), or written direction (e.g., through written direction from, for example, a veterinarian or other health professional (e.g., scripts), sales professional or organization (e.g., through, for example, marketing brochures, pamphlets, or other instructive paraphernalia), written media (e.g., internet, electronic mail, or other computer-related media)), and/or packaging associated with the composition (e.g., a label present on a container holding the composition). As used herein, “written” means through words, pictures, symbols, and/or other visible descriptors. Such information need not utilize the actual words used herein, for example, “respiratory”, “mammal”, “human”, or “treatment”, but rather use of words, pictures, symbols, and the like conveying the same or similar meaning are contemplated within the scope of this invention.

The compositions described herein may be orally administered in any convenient form, non-limiting examples of which include, for example, a capsule, tablet, including enteric coated and sustained-release forms, suspension, confectionery such as a gum or soft ‘gummie’, powders, including powders which are suitable for admixture with, dissolving, and/or dispersing in a liquid, non-limiting examples of which include water, milk, juice, hot and/or cold beverage, hot chocolate, cold cereal, hot cereal, yogurt, ice cream or the like. In one embodiment herein, the composition is a powder. The compositions described herein may be used as a supplement to ordinary diet (e.g., a dietary supplement) or may also serve as food for the mammal (e.g., used in a yogurt or other dairy product).

Administration may be on an as-needed or as-desired basis, for example, once-monthly, once-weekly, or daily, including multiple times daily, to arrive at a total daily dose or amount of a given component, whether administered every day, one day per week, one day per month, or on a given day as needed. When utilized as a supplement to ordinary diet, the composition may be administered directly to the mammal (e.g., a capsule or tablet) or otherwise contacted with or admixed with food (e.g., powder mixed with yogurt, juice or milk). The amount of composition utilized may be dependent on a variety of factors, including the health status of the mammal, age, gender, or other like factors of ordinary consideration.

In one embodiment the mammal is a human child between the ages of 2 and 18, alternatively between the ages of 3 and 12 and alternatively between the ages of 6 and 9 years of age.

The methods of the invention utilize the compositions described herein and include administration of at least about 1×10⁴ CFU of probiotic strain of bacteria, alternatively from about 10⁴ to about 10¹⁴ CFU, alternatively from about 10⁶ to about 10¹² CFU and alternatively from about from about 10⁸ to about 10¹¹ CFU of probiotic strain of bacteria, per unit dose of the composition. Alternatively the methods of the invention include administration of at least about 1×10⁴, alternatively at least about 1×10⁹, alternatively at least about 1×10¹⁰, and alternatively at least about 5×10¹⁰ cells per day of the probiotic strain of bacteria, which can be administered in a single dose or a plurality of doses

### Kits

Another embodiment, the invention can comprise a kit. The kit can comprise one or more individual doses of the compositions, for example one or more sachets containing a powdered composition and/or one confectioneries containing the composition; instructions, written, and/or pictorial as described above for methods of orally administering the compositions; one or more individual doses and/or samples of at least one additional component, for example a respiratory composition, one or more prebiotics, fibers, vitamins, minerals, metals, elements, plant-derived components, fungus-derived components, carotenoids, and/or antioxidants, as described above, if the doses of the compositions do not contain these components. The kit can further comprise a coupon, rebate or advertisement. The kit can also further comprise a toy, prize, game or educational material such as a booklet or coloring book. If the kit contains doses of several components packaged separately, the components can be packaged in dosage groups within the kit, i.e. one of each component to be taken together as a dose can be grouped together within the kit, for example by day.

### Methods of Making

The compositions disclosed herein can be delivered in any suitable pharmaceutical, supplement, food, and other form for ingestion. Conventional methods of manufacture of these forms are known in the art. Non-limiting examples include:

### Powder-Filled Hard Capsules

Powder ingredients including the probiotics, additional components, excipients and process aids are thoroughly mixed in a V-blender (20 cu. ft, stainless steel V-blender mixer, Patterson Kelly). The powder mixture is then filled into hard capsules (Vcaps two-piece hydroxypropyl methyl cellulose capsules, Capsugel) using an automated capsule filler (Model GKF 2500 S high speed capsule filler, Bosch). When certain vitamins, minerals, metals, elements and the like are included as additional components in capsule, tablet and powder forms, the actual amounts of these many of these components, in grams per unit dose, are often extremely small, and make the individual components difficult to handle, measure and process. Therefore such components are commonly prepared or purchased as a premix in or on a carrier such as sucrose or lactose. The premix of such components, in or on the carrier, is mixed with the probiotics, excipients and process aids.

### Direct Compression Tablets

Powder ingredients including the probiotics, additional components such as vitamins, excipients, and process aids are thoroughly mixed in a V-blender (20 cu. ft, stainless steel V-blender mixer, Patterson Kelly). The powder is pressed into tablets using a high-speed tabletting machine (VGK-3000 Series double-sided rotary tablet press, Vanguard). When vitamins, minerals, metals, elements and the like are included as additional components in such tablet forms, the actual amounts of these components, in grams per unit dose, are often extremely small, and make the individual components difficult to handle, measure and process. Therefore such components are commonly prepared or purchased as a premix in or on a carrier such as sucrose or lactose. The premix of such components, in or on the carrier, is mixed with the probiotics, excipients and process aids.

### Coated Tablets

Tablets, for example such as made by the method described above, are coated with a methacrylic acid co-polymer type C enteric coating using an aqueous suspension containing opacifier (such as titanium dioxide), anti-foaming agent (such as silicone) and plasticizer (as described above). Tablets are poured into the fully perforated pan of a Freund Vector VHC-48 Hi-Coater tablet coating machine. The coating suspension is applied using an atomizing spray nozzle with a pan speed of 8 rpm and an inlet air temperature of 55° C.

### Powder-Filled Pouches

Powder ingredients including the probiotics, additional components to enhance performance, excipients, and process aids are thoroughly mixed in a V-blender (20 cu. ft, stainless steel V-blender mixer, Patterson Kelly). The powder is filled into foil laminate pouches using a vertical form fill seal machine (FFS 0520, Sikri Packaging). When vitamins, minerals, metals, elements and the like are included as additional components in such powder forms, the actual amounts of these components, in grams per unit dose, are often extremely small, and make the individual components difficult to handle, measure and process. Therefore such components are commonly prepared or purchased as a premix in or on a carrier such as sucrose or lactose. The premix of such components, in or on the carrier, is mixed with the probiotics, excipients and process aids.

### Example COMPOSITIONS

Below are illustrated various non-limiting examples of preparations of compositions of the present invention. The following examples are for illustrative purposes only and further describe and demonstrate embodiments within the scope of the present invention. They are given for the purpose of illustration only, and are not to be construed as limitations of the present invention.

### Example 1

A non-limiting composition for a unit dose of a powder-filled hard capsule has the following components in the indicated amounts, and can be made by the method described above for making powder-filled hard capsules:

Lactobacillus acidophilus CBS 116411, (LAFTI® L10, DSM), freeze-dried powder, 10% by weight of the composition, and which contains 1×10⁹ CFU of bacteria per unit dose Microcrystalline cellulose (a filler) (Avicel® PH112, FMC), 89% by weight of the composition Magnesium stearate (a lubricant) (MF-2, Peter Greven), 1% by weight of the composition

### Example 2

A non-limiting composition for a unit dose of a direct compression tablet has the following components in the indicated amounts, and can be made by the method described above for making direct compression tablets:

Lactobacillus acidophilus CBS 116411, (LAFTI® L10, DSM), freeze-dried powder, 10% by weight of the composition, and which contains 1×10⁸ CFU of bacteria per unit dose Vitamin/Mineral Premix, 3% by weight of the composition
Premix contains and delivers the following vitamin/mineral content, per unit dose, in a lactose carrier:
Iron, 3.5 mg
Vitamin A, 648 IU
Iodine, 37.5 mcg
   Microcrystalline cellulose (a compression aid) (Avicel® PH112, FMC), 80% by weight of the composition
   Crospovidone (a disintegrant) (Kollidon® CL, BASF), 5% by weight of the composition Magnesium stearate (a lubricant) (MF-2, Peter Greven), 2% by weight of the composition

### Example 3

A non-limiting composition for a unit dose of a powder-filled pouch has the following components in the indicated amounts, and can be made by the method described above for making powder filled pouches:

Lactobacillus acidophilus CBS 116411, (LAFTI® L10, DSM), freeze-dried powder, 10% by weight of the composition, and which contains and 1×10¹⁰ CFU of bacteria per unit dose Vitamin/Mineral Premix, 27% by weight of the composition
Premix contains and delivers the following vitamin/mineral content, per unit dose, in a sucrose carrier:
Vitamin C, 60 mg
Iron, 3.5 mg
Vitamin A, 648 IU
Iodine, 37.5 mcg
Zinc, 3.75 mg
Vitamin E, 2.5 IU
Niacin (B-3), 2.5 mg
Pyridoxine HCL (B-6), 0.50 mg
Riboflavin (B-2), 0.455 mg
Folic Acid (B-9), 59.5 mcg
Cyanocobalamin (B-12), 0.50 mg
   Granulated sugar (a filler and sweetener) (Baker's Special, United Sugars), 57% by weight of the composition
   Crospovidone (a disintegrant) (Kollidon® CL, BASF), 5% by weight of the composition
   Magnesium stearate (a lubricant) (MF-2, Peter Greven), 1% by weight of the composition

### Example 4

A non-limiting composition for a unit dose of a powder-filled hard capsule has the following components in the indicated amounts, and can be made by the method described above for making a powder-filled hard capsule:

Bifidobacterium lactis CBS 118529 (LAFTI®Q L94, DSM), freeze-dried powder, 10% by weight of the composition, and which contains 1×10⁹ CFU of bacteria per unit dose
Microcrystalline cellulose (a filler) (Avicel® PH112, FMC), 89% by weight of the composition
Magnesium stearate (a lubricant) (MF-2, Peter Greven), 1% by weight of the composition

### Example 5

A non-limiting composition for a unit dose of a direct compression tablet has the following components in the indicated amounts, and can be made by the method described above or making a direct compression tablet:

Bifidobacterium lactis CBS 118529 (LAFTI® L94, DSM), freeze-dried powder, 10% by weight of the composition, and which contains 1×10⁷ CFU of bacteria per unit dose
Vitamin/Mineral Premix, 3% by weight of the composition
Premix contains and delivers the following vitamin/mineral content, per dose, in a lactose carrier:
Iron, 3.5 mg
Vitamin A, 648 IU
Iodine, 37.5 mcg
   Microcrystalline cellulose (a compression aid) (Avicel® PH112, FMC), 80% by weight of the composition
   Crospovidone (a disintegrant) (Kollidon® CL, BASF), 5% by weight of the composition
   Magnesium stearate (a lubricant) (MF-2, Peter Greven), 2% by weight of the composition

### Example 6

A non-limiting composition for a unit dose of a power-filled pouch has the following components in the indicated amounts, and can be made by the method described above for making powder-filled pouches:

Bifidobacterium lactis CBS 118529 (LAFTI® L94, DSM), freeze-dried powder, 10% by weight of the composition, and which contains 1×10¹¹ CFU of bacteria per unit dose
Vitamin/Mineral Premix, 27% by weight of the composition
Premix contains and delivers the following vitamin/mineral content, per dose, in a sucrose carrier:
Vitamin C, 60 mg
Iron, 3.5 mg
Vitamin A, 648 IU
Iodine, 37.5 mcg
Zinc, 3.75 mg
Vitamin E, 2.5 IU
Niacin (B-3), 2.5 mg
Pyridoxine HCL (B-6), 0.50 mg
Riboflavin (B-2), 0.455 mg
Folic Acid (B-9), 59.5 mcg
Cyanocobalamin (B-12), 0.50 mg
   Granulated sugar (a filler and sweetener) (Baker's Special, United Sugars), 57% by weight of the composition
   Crospovidone (a disintegrant) (Kollidon® CL, BASF), 5% by weight of the composition
   Magnesium stearate (a lubricant) (MF-2, Peter Greven), 1% by weight of the composition

### Example 7

A non-limiting composition for a unit dose of a powder-filled hard capsule has the following components in the indicated amounts, and can be made by the method described above for making powder-filled hard capsules:

Lactobacillus acidophilus CBS 116411 (LAFTI® L10, DSM), freeze-dried powder, 5% by weight of the composition and Bifidobacterium lactis CBS 118529 (LAFTI® L94, DSM), freeze-dried powder, 5% by weight of the composition, which, when combined, contains 1×10⁹ CFU of bacteria per unit dose
Microcrystalline cellulose (a filler) (Avicel® PH112, FMC), 89% by weight of the composition
Magnesium stearate (a lubricant) (MF-2, Peter Greven), 1% by weight of the composition

### Example 8

A non-limiting composition for a unit dose of a direct compression tablet has the following components in the indicated amounts, and can be made by the method described above for making direct compression tablets:

Lactobacillus acidophilus CBS 116411 (LAFTI® L10, DSM), freeze-dried powder, 5% by weight of the composition and Bifidobacterium lactis CBS 118529 (LAFTI® L94, DSM), freeze-dried powder, 5% by weight of the composition, which, when combined, contains 1×10⁶ CFU of bacteria per unit dose
Vitamin/Mineral Premix, 3% by weight of the composition
Premix contains and delivers the following vitamin/mineral content, per unit dose, in a lactose carrier:
Iron, 3.5 mg
Vitamin A, 648 IU
Iodine, 37.5 mcg
   Microcrystalline cellulose (a compression aid) (Avicel® PH112, FMC), 80% by weight of the composition
   Crospovidone (a disintegrant) (Kollidon® CL, BASF), 5% by weight of the composition
   Magnesium stearate (a lubricant) (MF-2, Peter Greven), 2% by weight of the composition

### Example 9

A non-limiting composition for a unit dose of a powder-filled pouch has the following components in the indicated amounts and can be made by the method described above for making powder-filled pouches:

Lactobacillus acidophilus CBS 116411 (LAFTI® L10, DSM), freeze-dried powder, 5% by weight of the composition and Bifidobacterium lactis CBS 118529 (LAFTI® L94, DSM), freeze-dried powder, 5% by weight of the composition which, when combined, contains 1×10¹¹ CFU of bacteria per unit dose
Vitamin/Mineral Premix, 27% by weight of the composition
Premix contains and delivers the following vitamin/mineral content, per unit dose, in a sucrose carrier:
Vitamin C, 60 mg
Iron, 3.5 mg
Vitamin A, 648 IU
Iodine, 37.5 mcg
Zinc, 3.75 mg
Vitamin E, 2.5 IU
Niacin (B-3), 2.5 mg
Pyridoxine HCL (B-6), 0.50 mg
Riboflavin (B-2), 0.455 mg
Folic Acid (B-9), 59.5 mcg
Cyanocobalamin (B-12), 0.50 mg
   Granulated sugar (a filler, and sweetener) (Baker's Special, United Sugars), 57% by weight of the composition
   Crospovidone (a disintegrant) (Kollidon® CL, BASF), 5% by weight of the composition
   Magnesium stearate (a lubricant) (MF-2, Peter Greven), 1% by weight of the composition

### Example 10

A non-limiting composition for a unit dose of a powder-filled hard capsule has the following components in the indicated amounts and can be made by the method described above for making powder-filled hard capsules:

Bifidobacterium bifidum freeze-dried powder, 10% by weight of the composition, which contains 1×10¹¹ CFU of bacteria per unit dose
Microcrystalline cellulose (a filler) (Avicel® PH112, FMC), 89% by weight of the composition
Magnesium stearate (a lubricant) (MF-2, Peter Greven), 1% by weight of the composition

### Example 11

A non-limiting composition for a unit dose of a direct compression tablet has the following components in the indicated amounts and can be made by the method described above for making direct compression tablets:

Bifidobacterium longum freeze-dried powder, 10% by weight of the composition, which contains 1×10⁹ CFU of bacteria per unit dose
Vitamin/Mineral Premix, 3% by weight of the composition
Premix contains and delivers the following vitamin/mineral content, per unit dose, in a lactose carrier:
Iron, 3.5 mg
Vitamin A, 648 IU
Iodine, 37.5 mcg
   Microcrystalline cellulose (a compression aid) (Avicel® PH112, FMC), 80% by weight of the composition
   Crospovidone (a disintegrant) (Kollidon® CL, BASF), 5% by weight of the composition
   Magnesium stearate (a lubricant) (MF-2, Peter Greven), 2% by weight of the composition

### Example 12

A non-limiting composition for a unit dose of a powder-filled pouch has the following components in the indicated amounts and can be made by the method described above for making powder-filled pouches:

Bifidobacterium infantis freeze-dried powder, 10% by weight of the composition which contains 1×10¹⁰ CFU of bacteria per unit dose
Vitamin/Mineral Premix, 27% by weight of the composition
Premix contains and delivers the following vitamin/mineral content, per unit dose, in a sucrose carrier:
Vitamin C, 60 mg
Iron, 3.5 mg
Vitamin A, 648 IU
Iodine, 37.5 mcg
Zinc, 3.75 mg
Vitamin E, 2.5 IU
Niacin (B-3), 2.5 mg
Pyridoxine HCL (B-6), 0.50 mg
Riboflavin (B-2), 0.455 mg
Folic Acid (B-9), 59.5 mcg
Cyanocobalamin (B-12), 0.50 mg
   Granulated sugar (a filler, and sweetener) (Baker's Special, United Sugars), 57% by weight of the composition
   Crospovidone (a disintegrant) (Kollidon® CL, BASF), 5% by weight of the composition Magnesium stearate (a lubricant) (MF-2, Peter Greven), 1% by weight of the composition

### Example 13

A non-limiting composition for a unit dose of a direct compression tablet has the following components in the indicated amounts and can be made by the method described above for making direct compression tablets:

Bifidobacterium longum freeze-dried powder, 5% by weight of the composition and Lactobacillus acidophilus freeze-dried powder, 5% by weight of the composition, which, when combined, contains 1×10⁷ CFU of bacteria per unit dose
Vitamin/Mineral Premix, 3% by weight of the composition
Premix contains and delivers the following vitamin/mineral content, per unit dose, in a lactose carrier:
Iron, 3.5 mg
Vitamin A, 648 IU
Iodine, 37.5 mcg
   Microcrystalline cellulose (a compression aid) (Avicel® PH112, FMC), 80% by weight of the composition
   Crospovidone (a disintegrant) (Kollidon® CL, BASF), 5% by weight of the composition Magnesium stearate (a lubricant) (MF-2, Peter Greven), 2% by weight of the composition

### Example 14

A non-limiting composition for a unit dose of an enteric-coated, direct compression tablet has the components in the indicated amounts, and can be made by the methods described above for direct compression tablets and coated tablets:

Lactobacillus acidophilus CBS 116411 (LAFTI® L10, DSM) freeze-dried powder, 10% by weight of the composition, and which contains 1×10⁶ CFU of bacteria per unit dose
Vitamin/Mineral Premix, 3% by weight of the composition
Premix contains the following vitamin/mineral content, per unit dose, in a lactose carrier:
Iron, 3.5 mg
Vitamin A, 648 IU
Iodine, 37.5 mcg
   Microcrystalline cellulose (a compression aid) (Avicel® PH112, FMC), 78% by weight of the composition
   Magnesium stearate (a lubricant) (MF-2, Peter Greven), 2% by weight of the composition
   Methacrylic acid co-polymer type C (Acryl-EZE®, Colorcon) 7% by weight of the composition

### Example 15

A non-limiting composition for a unit dose of a powder-filled hard capsule has the following components in the indicated amounts, and can be made by the methods described above for making powder-filled hard capsules:

Heat-treated Lactobacillus acidophilus CL-92, freeze-dried powder, 10% by weight of the composition, and which contains 2×10¹⁰ cells of bacteria (Calpis Co., Ltd., Japan)
Superfine extract of shiitake mushroom, 10% by weight of the composition, (which provides 15 mg of extract) (Aginomoto, Japan)
Microcrystalline cellulose (a filler) (Avicel® PH112, FMC) 79% by weight of the composition
Magnesium stearate (a lubricant) (MF-2, Peter Greven) 1% by weight of the composition

### Example 16

A non-limiting composition for a unit dose of a powder-filled pouch has the following components in the indicated amounts and can be made by the method described above for making powder-filled pouches:

Heat-treated Lactobacillus acidophilus CL-92 freeze-dried powder, 10% by weight of the composition which contains 5×10¹⁰ cells of bacteria (Calpis Col, Ltd., Japan)
Superfine extract of shiitake mushroom, 10% by weight of the composition, (which provides 15 mg of extract) (Ajinomoto, Japan)
Vitamin/Mineral Premix, 27% by weight of the composition
Premix contains and delivers the following vitamin/mineral content, per unit dose, in a sucrose carrier:
Vitamin C, 60 mg
Iron, 3.5 mg
Vitamin A, 648 IU
Iodine, 37.5 mcg
Zinc, 3.75 mg
Vitamin E, 2.5 IU
Niacin (B-3), 2.5 mg
Pyridoxine HCL (B-6), 0.50 mg
Riboflavin (B-2), 0.455 mg
Folic Acid (B-9), 59.5 mcg
Cyanocobalamin (B-12), 0.50 mg
   Granulated sugar (a filler, and sweetener) (Baker's Special, United Sugars), 47% by weight of the composition
   Crospovidone (a disintegrant) (Kollidon® CL, BASF), 5% by weight of the composition
   Magnesium stearate (a lubricant) (MF-2, Peter Greven), 1% by weight of the composition

### Example 17

A non-limiting composition for a unit dose of a powder-filled pouch has the following components in the indicated amounts and can be made by the method described above for making powder-filled pouches:

Heat-treated Lactobacillus acidophilus CL-92 freeze-dried powder, 10% by weight of the composition which contains 5×10¹⁰ cells of bacteria (Calpis Co., Ltd., Japan)
Superfine beta-1,3-glucan, 10% by weight of the composition, (which provides 50 mg of extract) (Ajinomoto, Japan)
Granulated sugar (a filler, and sweetener) (Baker's Special, United Sugars), 74% by weight of the composition
Crospovidone (a disintegrant) (Kollidon® CL, BASF), 5% by weight of the composition
Magnesium stearate (a lubricant) (MF-2, Peter Greven), 1% by weight of the composition

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition for enhancing immune response to a respiratory condition in a mammal comprising a therapeutic amount of a strain of probiotic bacteria and a therapeutic amount of an additional component.

2. The composition of claim 1 wherein said probiotic bacteria is selected from the group consisting of Streptococcus lactis, Streptococcus cremoris, Streptococcus diacetylactis, Streptococcus thermophilus, Lactobacillus bulgaricus, Lactobacillus acidophilus, Lactobacillus helveticus, Lactobacillus bifidus, Lactobacillus casei, Lactobacillus lactis, Lactobacillus plantarum, Lactobacillus rhamnosus, Lactobacillus delbruekii, Lactobacillus thermophilus, Lactobacillus fermentii, Lactobacillus salivarius, Lactobacillus reuteri, Lactobacillus brevis, Lactobacillus paracasei, Lactobacillus gasseri, Pediococcus cerevisiae, Bifidobacterium longum, Bifidobacterium infantis, Bifidobacterium adolescentis, Bifidobacterium bifidum, Bifidobacterium animalis, Bifidobacterium pseudolongum, Bifidobacterium thermophilum, Bifidobacterium lactis, Bifidobacterium bulgaricus, Bifidobacterium breve, Bifidobacterium subtilis, strains of the genera Bacillus, Bacteroides, Enterococcus and Leuconostoc, and combinations thereof.

3. The composition of claim 2 wherein said strain of probiotic bacteria comprises a strain of Lactobacillus selected from the group consisting of Lactobacillus acidophilus and Lactobacillus fermentum, and combinations thereof.

4. The composition of claim 3 comprising from about 1.0×10⁴ CFU to about 1.0×10¹⁴ CFU of said Lactobacillus per unit dose of said composition.

5. The composition of claim 4 comprising from about 1.0×10⁸ CFU to about 1.0×10¹¹ CFU of said Lactobacillus per unit dose of said composition.

6. The composition of claim 3 comprising at least about 1×10⁹ cells of said Lactobacillus per daily dose of said composition.

7. The composition of claim 2 wherein said probiotic strain of bacteria comprises a strain of Bifidobacterium lactis.

8. The composition of claim 7 comprising from about 1.0×10⁴ CFU to about 1.0×10¹⁴ CFU of said Bifidobacterium per unit dose of said composition.

9. The composition of claim 8 comprising from about 1.0×10⁸ CFU to about 1.0×10¹¹ CFU of said Bifidobacterium per unit dose of said composition.

10. The composition of claim 1 further comprising a therapeutic amount of an additional component selected from the group consisting of an additional probiotic strain of bacteria; prebiotics; fiber; vitamins; minerals; metals; elements; plant-derived components; fungal-derived components; carotenoids; anti-oxidants and combinations thereof.

11. The composition of claim 10 wherein said fungal-derived component comprises superfine particles of shiitake mushroom.

12. The composition of claim 11 comprising from about 0.01 mg to about 50 mg of said superfine particles of shiitake mushroom per daily dose of the composition.

13. The composition of claim 10 wherein said additional component comprises superfine particles of beta-glucan.

14. The composition of claim 13 comprising from about 0.01 mg to about 50 g of said superfine particles of beta-glucan per daily dose of said composition.

15. The composition of claim 10 wherein said strain of probiotic bacteria comprises a strain of Lactobacillus acidophilus and said fungal-derived component comprises superfine particles of shiitake mushroom.

16. The composition of claim 10 wherein said strain of probiotic bacteria comprises a strain of Lactobacillus acidophilus and said additional component comprises superfine particles of beta-glucan.

17. The composition of claim 2 comprising a therapeutic amount of a strain of Lactobacillus, and a therapeutic amount of a strain of Bifidobacterium.

18. The composition of claim 17 comprising a therapeutic amount of a strain of Lactobacillus, a therapeutic amount of a strain of Bifidobacterium, and a therapeutic amount of iron, iodine, and Vitamin A.

19. A method of enhancing immune response to a respiratory condition in a mammal comprising orally administering to said mammal a composition comprising a therapeutic amount of a strain of probiotic bacteria and a therapeutic amount of an additional component.

20. The method of claim 20 wherein said probiotic bacteria is selected from the group consisting of Streptococcus lactis, Streptococcus cremoris, Streptococcus diacetylactis, Streptococcus thermophilus, Lactobacillus bulgaricus, Lactobacillus acidophilus, Lactobacillus helveticus, Lactobacillus bifidus, Lactobacillus casei, Lactobacillus lactis, Lactobacillus plantarum, Lactobacillus rhamnosus, Lactobacillus delbruekii, Lactobacillus thermophilus, Lactobacillus fermentii, Lactobacillus salivarius, Lactobacillus reuteri, Lactobacillus brevis, Lactobacillus paracasei, Lactobacillus gasseri, Pediococcus cerevisiae, Bifidobacterium longum, Bifidobacterium infantis, Bifidobacterium adolescentis, Bifidobacterium bifidum, Bifidobacterium animalis, Bifidobacterium pseudolongum, Bifidobacterium thermophilum, Bifidobacterium lactis, Bifidobacterium bulgaricus, Bifidobacterium breve, Bifidobacterium subtilis, stains of the genera Bacillus, Bacteroides, Enterococcus and Leuconostic, and combinations thereof.

21. The method of claim 20 wherein said probiotic bacteria comprises a strain of Lactobacillus selected from the group consisting of Lactobacillus acidophilus and Lactobacillus fermentum.

22. The method of claim 21 wherein said composition comprises from about 1.0×10⁴ CFU to about 1.0×10¹⁴ CFU of said Lactobacillus per unit dose of said composition.

23. The method of claim 22 wherein said composition comprises from about 1.0×10⁸ CFU to about 1.0×10¹¹ CFU of said Lactobacillus per unit dose of said composition.

24. The method of claim 21 wherein said composition comprises at least about 1×10⁹ cells of said Lactobacillus per daily dose of said composition.

25. The method of claim 20 wherein said probiotic strain of bacteria comprises a strain of Bifidobacterium lactis.

26. The method of claim 25 wherein said composition comprises from about 1.0×10⁴ CFU to about 1.0×10¹⁴ CFU of said Bifidobacterium per unit dose of said composition.

27. The method of claim 26 wherein said composition comprises from about 1.0×10⁸ CFU to about 1.0×10¹¹ CFU of said Bifidobacterium per unit dose of said composition.

28. The method of claim 20 wherein said additional component is selected from the group consisting of an additional probiotic strain of bacteria; prebiotics; fiber; vitamins; minerals; metals; elements; plant-derived components; fungal-derived components; carotenoids; anti-oxidants and combinations thereof.

29. The method of claim 28 wherein said fungal-derived component comprises superfine particles of shiitake mushroom.

30. The method of claim 29 comprising from about 0.01 mg to about 50 mg of said superfine particles of shiitake mushroom per daily dose of said composition.

31. The method of claim 28 wherein said additional component comprises superfine particles of beta-glucan.

32. The method of claim 31 comprising from about 0.01 mg to about 50 g of said superfine particles of beta-glucan per daily dose of said composition.

33. The method of claim 28 wherein said strain of probiotic bacteria comprises a strain of Lactobacillus acidophilus and said fungal-derived component comprises superfine particles of shiitake mushroom.

34. The method of claim 28 wherein said strain of probiotic bacteria comprises a strain of Lactobacillus acidophilus and said additional component comprises superfine particles of beta-glucan.

35. The method of claim 19 wherein said mammal is a human child from about 2 to about 18 years of age.

36. The method of claim 19 wherein said composition is administered one or more times daily.

37. The method of claim 19 further comprising mixing said composition with a hot, warm, or cold beverage or food selected from the group consisting of water, milk, juice, yogurt, ice cream, cold cereal, hot cereal, coffee, tea, hot chocolate and combinations thereof.

38. The method of claim 19 wherein said composition comprises a form selected from the group consisting of tablet, enteric coated tablet, sustained release tablet, capsule, enteric coated capsule, sustained-release capsule, powder, confectionery, and combinations thereof.

39. The method of claim 28 comprising orally administering to said mammal a composition comprising a therapeutic amount of a strain of Lactobacillus and a therapeutic amount of a strain of Bifidobacterium.

40. The method of claim 39 comprising administering at least about 1.0×10⁴ CFU of said Lactobacillus per dose of said composition.

41. The method of claim 39 comprising administering at least about 1.0×10⁴ CFU of said Bifidobacterium per dose of said composition.

42. The method of claim 28 comprising orally administering to said mammal a therapeutic amount of a strain of Lactobacillus, a therapeutic amount of a strain of Bifidobacterium, and a therapeutic amount of iron, iodine, and vitamin A.

43. A kit comprising compositions for enhancing immune response, said kit comprising:
a. at least one dose of a composition comprising a therapeutic amount of a probiotic strain of bacteria;
b. at least one dose of a therapeutic amount of an additional component; and
c. printed material or instructions for administering said compositions.

44. The kit of claim 42 wherein said additional component selected from the group consisting of an additional probiotic strain of bacteria; prebiotics; fiber; vitamins; minerals; metals; elements; plant-derived components; fungal-derived components; carotenoids; anti-oxidants and combinations thereof; wherein said at least one additional component is combined with said strain of probiotic bacteria in the same composition or is packaged as one or more separate components of said kit.

45. The kit of claim 43 wherein said at least one additional component comprises superfine particles of shiitake mushroom.

46. The kit of claim 43 wherein said at least one additional component comprises superfine particles of beta-glucan.

47. The kit of claim 43 wherein said at least one additional component comprises at least one vitamin.
